(19) United States
(12) Patent Application Publication
(10) Pub. No.: US 2023/0158047 A1
(43) Pub. Date: May 25, 2023

(54) METHODS AND COMPOSITIONS FOR TREATING PROSTATE CANCER

(71) Applicant: Tavanta Therapeutics Hungary Incorporated, Budapest (HU)

(72) Inventors: Richard Balázs Kárpáti, Budapest (HU); Zsolt Ötvös, Budapest (HU); Tamás Jordán, Budapest (HU); Andrea Ujhelyi, Budapest (HU); Orsolya Basa-Dénes, Budapest (HU); Tamás Solymosi, Budapest (HU); Hristos Glavinas, Budapest (HU); Dominic P. Capone, Abington, PA (US); Elizabeth Manning Duus, Cape Coral, FL (US); John E. Friend,, II, Colts Neck, NJ (US)

(73) Assignee: Tavanta Therapeutics Hungary Incorporated, Budapest (HU)

(21) Appl. No.: 17/919,170

(22) PCT Filed: Apr. 15, 2021
(86) PCT No.: PCT/IB2021/053116

§ 371 Date: Oct. 14, 2022

Related U.S. Application Data
(60) Provisional application No. 63/011,023, filed on Apr. 16, 2020.

(51) Int. Cl. A61K 31/58; A61K 9/20; A61K 9/00; A61J 3/10; A23L 33/10

(52) U.S. Cl. CPC A61K 31/58; A61K 9/2027; A61K 9/2013; A61K 9/2018; A61K 9/2054; A61K 9/2009; A61K 9/2031; A61K 9/0056; A61J 3/10; A23L 33/10

(57) ABSTRACT

Methods and compositions for treating prostate cancer are described herein. More particularly, the methods for treating prostate cancer comprise administering abiraterone acetate in the form of a solid dosage form for oral administration wherein said solid dosage form for oral administration is capable of rapid reconstitution or disintegration in the oral cavity or an aliquot of liquid prior to ingestion, suitably administered in combination with a steroid.

### FIELD OF THE INVENTION

Methods and compositions for treating prostate cancer are described herein. More particularly, the methods for treating prostate cancer comprise administering abiraterone acetate in the form of a solid dosage form for oral administration wherein said solid dosage form for oral administration is capable of rapid reconstitution or disintegration in the oral cavity or an aliquot of liquid prior to ingestion, suitably administered in combination with a steroid. Furthermore, disclosed is a composition comprising abiraterone acetate and methods of formulating and manufacturing said solid dosage form for oral administration according to the invention.

### BACKGROUND OF THE INVENTION

Abiraterone acetate is 3β-acetoxy-17-(3-pyridyl)-androsta-5,16-diene that is administered orally. It is rapidly converted in vivo to abiraterone, a selective, irreversible inhibitor of cytochrome P450 17α (17α-hydroxylase/C₁₇₋₂₀ lyase; CYP17), an enzyme that is key in the production of androgens in all sites, including the testes and adrenal glands.

Abiraterone acetate is currently marketed under the trade name Zytiga® for the treatment of metastatic castration-resistant prostate cancer (CRPC) and metastatic high-risk castration-sensitive prostate cancer (CSPC), in combination with prednisone. Zytiga® is presented as 250 mg immediate release tablets containing abiraterone acetate as active substance. The excipients used in the formulation of Zytiga® are lactose monohydrate, microcrystalline cellulose, croscarmellose sodium, povidone, sodium lauryl sulfate, magnesium stearate and colloidal silicon dioxide. The recommended dose of Zytiga® is 1000 mg administered orally once daily.

Zytiga® decreases serum testosterone and other androgens to levels lower than those achieved by the use of LHRH analogues alone or by orchiectomy. This results from the selective inhibition of the CYP17 enzyme required for androgen biosynthesis. Prostate specific antigen (PSA) serves as a biomarker in patients with prostate cancer. In a phase 3 clinical study of patients who failed prior chemotherapy with taxanes, 38% of patients treated with abiraterone acetate, versus 10% of patients treated with placebo, had at least a 50% decline from baseline in PSA levels.

Following oral administration of Zytiga® in the fasting state, the time to reach maximum plasma abiraterone concentration is approximately 2 hours. Administration of Zytiga® with food, compared with administration in a fasted state, results in up to a 10-fold (AUC) and up to a 17-fold (Cₘₐₓ) increase in mean systemic exposure of abiraterone, depending on the fat content of the meal. Given the normal variation in the content and composition of meals, taking Zytiga® with meals has the potential to result in highly variable exposures.

The 10-fold food effect observed also shows that the oral bioavailability of abiraterone is below 10% when Zytiga® is administered in the fasted state. Inter-subject variability has been shown to be approximately 79% for Cₘₐₓ and 64% for AUC₀₋₂₄ after multiple day dosing.

Steady state is achieved within eight days following once-daily dosing of 1000 mg, with approximately 2-fold higher exposure at steady state (steady state AUC) compared to when the same dose is administered as a single dose. Inter-subject variability has been shown to be approximately 79% for Cₘₐₓ and 64% for AUC0-24 after multiple day dosing.

The low bioavailability, significant food effect and the highly variable nature of abiraterone pharmacokinetics (PK) results in known efficacy and potential safety issues. It has been shown that patients who exhibited primary resistance to 1,000 mg Zytiga® had lower plasma abiraterone concentrations when compared to responders. Furthermore, it was suggested, that at higher plasma concentrations abiraterone also blocks 3β-hydroxysteroid dehydrogenase (3βHSD), an enzyme that is required for the synthesis of biologically active androgens. Higher abiraterone acetate doses were shown to decrease CRPC xenograft growth in DHEA-supplemented mice. This second mode of action might be exploited to reverse resistance to CYP17A1 inhibition at the standard abiraterone acetate dose by dose-escalation or simply by administration with food to increase drug exposure. However, based on the extent of variability in exposure when abiraterone acetate is taken with a high-fat food content, and given the normal variations in the content and composition of meals that cannot be controlled outside of the clinical trial setting, the FDA has requested that the product labeling reflect that abiraterone has to be administered 2 hours before meals.

In order to overcome the problems associated with prior conventional abiraterone acetate formulations and available drug delivery systems, novel solid dosage forms for oral administration, which are capable of rapid reconstitution or disintegration in the oral cavity or an aliquot of liquid prior to ingestion have been developed which are characterized by a reduced food effect, improved pharmacokinetic profile, reduced dose and reduced variability as compared to the commercial preparation Zytiga. The novel formulation is suitably administered as a homogeneous suspension, therefore, patients with dysphagia can also take it with ease.

### BRIEF DESCRIPTION OF THE INVENTION

In a first aspect of the invention there is provided, a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion.

In a second aspect of the invention there is provided, a solid dosage form for oral administration comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, comprising:
(a) 1-20% abiraterone acetate,
(b) 5-60% water soluble polymer,
(c) 35-90% fillers and/or diluents,
(d) 5-20% binders,
(e) 5-45% disintegrants,
(f) 0.1-5% lubricants,
(g) 0.1-5% antiadherents,
(h) 0.1-5% glidants,
(i) 0.01-2% antioxidants, and
(j) 0.05-5% flavouring agents and/or sweeteners.

In a third aspect of the invention there is provided, a method of producing a solid dosage form for oral administration comprising abiraterone acetate, wherein said solid dosage form for oral administration is capable of rapid reconstitution or disintegration in the oral cavity or an aliquot of liquid prior to ingestion, wherein said method comprising the steps of:
(a) providing abiraterone acetate;
(b) providing one or more water-soluble polymers, preferably polyvinylpyrrolidone, such as K12 or Kollidon 17PF, Kollidon 25, Kollidon 30 or Kollidon 90, most preferably a K12 grade;
(c) providing one or more antioxidants;
(d) processing (a), (b) and (c) by blending to produce powder blend comprising (a) to (c);
(e) processing the product of step (d) by an extrusion process to produce an extruded composition;
(f) processing the extruded composition of step (e) by size reduction such as milling, to produce a particulate or milled extrudate;
(g) blending the particulate or milled extruded composition of step (f) with one or more pharmaceutically acceptable excipients such as fillers, diluents, binders, disintegrants, lubricants, antiadherents, glidants, flavoring agents and sweeteners to produce a composition blend; and
(h) compressing the composition blend of step (g) into a solid dosage form such as via direct compression to form a tablet, or, preparing granules from the composition blend of step (g), via slugging and/or milling and/or roller compaction to produce such granules.

In a fourth aspect of the invention there is provided, a method of treating, reducing, inhibiting or controlling prostate cancer in a human patient in need thereof, comprising administering a therapeutically effective amount of a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion by said human patient.

### DETAILED DESCRIPTION OF THE INVENTION

In a preferred embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion.

In another embodiment of the invention, said stable, amorphous matrix exhibits one or more of the following: (i), X-ray amorphous character in the solid form; (ii), a glass transition when assessed via differential scanning calorimetry (DSC), and (iii), is stable for at least 3 months at 40° C., as demonstrated by a lack of significant decomposition and/or oxidation and/or crystallization.

In another preferred embodiment of the invention, said stable, amorphous matrix exhibits a glass transition temperature of at least 40° C., or at least 45° C., or at least 50° C. at the time of manufacture, with no melting peaks associated with said abiraterone acetate, preferably wherein said stable, amorphous complex is produced via hot melt extrusion (HME) or via lyophilisation.

In another preferred embodiment of the invention, said stable, amorphous matrix exhibits a glass transition temperature of at least 40° C., or at least 45° C., or at least 50° C. after at least 2 months at 40° C., preferably with no melting peaks associated with said abiraterone acetate.

In another embodiment of the invention, said stable, amorphous matrix comprises one or more water-soluble polymers selected from: polyvinylcaprolactam-polyvinyl acetate-polyethylene-glycol graft copolymers, poloxamers; polyvinylpyrrolidone, poly(acrylic acid), polyvinyl alcohol, ethylene glycol and vinyl alcohol graft copolymer, hydroxypropyl methylcellulose, hydroxypropylcellulose, hydroxyethylcellulose, hypromellose acetate succinate, polyethylene oxide, polyethylene-glycol, poly(2-Ethyl-2-oxazoline), poly(methyl vinyl ether/maleic anhydride), poly(maleic acid-co-methyl vinyl ether, polyoxylglycerides, D-α-tocopherol polyethylene glycol 1000 succinate, polyoxyethylene castor oil derivatives, polyoxyethylene sorbitan fatty acid esters, polylactic acid, poly(lactic-co-glycolic acid) and copolymers of vinylpyrrolidone and vinyl-acetate and poly(maleic acid-co-methyl-vinyl-ether), VIVAPHARM® PVP/VA 64 (a 6:4 linear random copolymer of N-vinylpyrrolidone and vinyl acetate), preferably polyvinylpyrrolidone, such as K12 or Kollidon 17PF, Kollidon 25, Kollidon 30 or Kollidon 90 (with molecular weights ranging from 2,500 to 1,000,000) most preferably a K12 grade (with a molecular weight of about 2,000 to 7,000, preferably about 2,500).

Plasdone (PVP) K-12 is a water-soluble polymer with low molecular weight and low viscosity. These features ensure the fast dispersion of the solid dosage form of the invention in liquid media or the oral cavity, which is extremely important in case of a water dispersible formulation. PVPs with higher molecular weights (Kollidon 17PF, Kollidon 25, Kollidon 30, Kollidon 90) and other polymers (Kollidon VA64, PVA, HPMC) may also be employed as the water-soluble polymer of the stable, amorphous complex. However, the reconstitution times are longer than in case of a Plasdone/PVP K-12 containing formulation.

In yet another embodiment of the invention, said stable, amorphous matrix comprises one or more antioxidants selected from: ascorbic acid, ascorbyl palmitate, butylated hydroxyanisole (BHA), butylated hydroxytoluene (BHA), hypophosphorous acid, monothioglycerol, potassium metabisulfite, propyl gallate, sodium bisulfite, sodium formaldehyde sulfoxylate, sodium metabisulfite, sodium sulfite, sodium thiosulfate and tocopherols, preferably BHA and/or BHT.

In yet another embodiment of the invention, said stable, amorphous matrix comprises BHT and BHA in order to protect said abiraterone acetate against oxidative degradation during a holt melt extrusion process and/or long-term storage.

In yet another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, and wherein said solid oral dosage form has a shelf-life of at least 12 months, preferably 24 months or longer, as demonstrated by an assay of between 90 to 110% of said label claim/content of said abiraterone acetate, when stored at room temperature, such as 25° C.

Alternatively, said solid oral dosage form has a shelf-life of at least 12 months, preferably 24 months or longer, as demonstrated by a maximum level of abiraterone acetate related substances of 1.0% by weight of said abiraterone acetate, when stored at room temperature, such as 25° C.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said one or more pharmaceutically excipients are selected from group consisting of: diluents or fillers, binders, disintegrants and dispersing agents, lubricants, glidants, antiadherents, surfactants, sweeteners and flavourings.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said one or more pharmaceutically excipients are selected from group consisting of: diluents or fillers, binders, disintegrants and dispersing agents, lubricants, glidants, antiadherents, surfactants, sweeteners and flavourings and wherein said diluents or fillers are selected from: pullulan, lactose (anhydrous), lactose monohydrate, mannitol, sucrose, glucose, plant cellulose, calcium carbonate, magnesium carbonate, magnesium oxide, microcrystalline cellulose, silicified microcrystalline cellulose, sorbitol, starch, pregelatinized starch, isomalt, anhydrous dibasic calcium phosphate, dibasic calcium phosphate dihydrate, calcium silicate, magnesium aluminium silicate, maltodextrin, dextrates.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said one or more pharmaceutically excipients are selected from group consisting of: diluents or fillers, binders, disintegrants and dispersing agents, lubricants, glidants, antiadherents, surfactants, sweeteners and flavourings and wherein said binders are selected from: pullulan, cellulose, methyl cellulose, microcrystalline cellulose, cellulose ethers such as hydroxypropyl cellulose, polyvinylpyrrolidone, polyethylene glycol, lactose, sucrose, mannitol, sorbitol and xylitol and their co-processed versions such as alpha-Lactose-monohydrate and cellulose or starch, urea crystals, sodium sulfate and calcium sulfate dihydrate.

In yet another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said one or more pharmaceutically excipients are selected from group consisting of: diluents or fillers, binders, disintegrants and dispersing agents, lubricants, glidants, antiadherents, surfactants, sweeteners and flavourings and wherein said disintegrants and dispersing agents are selected from: polyvinylpyrrolidone, crosslinked polyvinylpyrrolidone (crospovidone), polyvinylcaprolactam-polyvinyl acetate-polyethylene-glycol graft copolymers, carboxymethyl cellulose, crosslinked cellulose and its sodium salt (crosscarmellose), crosslinked sodium carboxymethyl cellulose, modified starch, sodium starch glycolate, crosslinked starch, crosslinked alginic acid and sodium starch glycolate, colloidal silicon dioxide, soy polysaccharides and sodium deoxycholate.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said one or more pharmaceutically excipients are selected from group consisting of: diluents or fillers, binders, disintegrants and dispersing agents, lubricants, glidants, antiadherents, surfactants, sweeteners and flavourings and wherein said lubricants are selected from: polyethylene glycol, calcium stearate, silica, talc, stearic acid, sodium stearyl fumarate, sodium lauryl sulfate, sodium benzoate, stearic acid and magnesium stearate.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said one or more pharmaceutically excipients are selected from group consisting of: diluents or fillers, binders, disintegrants and dispersing agents, lubricants, glidants, antiadherents, surfactants, sweeteners and flavourings and wherein said glidants are selected from: silica gel, colloidal silicon dioxide, fumed silica, talc and magnesium carbonate.

In yet another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said one or more pharmaceutically excipients are selected from group consisting of: diluents or fillers, binders, disintegrants and dispersing agents, lubricants, glidants, antiadherents, surfactants, sweeteners and flavourings and wherein said antiadherents are selected from: magnesium stearate, talc and starch.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said one or more pharmaceutically excipients are selected from group consisting of: diluents or fillers, binders, disintegrants and dispersing agents, lubricants, glidants, antiadherents, surfactants, sweeteners and flavourings, and wherein said surfactants are selected from: polysorbates such as Tweens, polysorbates, Sodium dodecyl sulfate (sodium lauryl sulfate), lauryl dimethyl amine oxide, docusate sodium, cetyl trimethyl ammonium bromide (CTAB), polyethoxylated alcohols, polyoxyethylene sorbitan, octoxynol, N,N-dimethyldodecylamine-N-oxide, hexadecyltrimethylammonium bromide, polyoxyl 10 lauryl ether, Brij, bile salts (sodium deoxycholate, sodium cholate), polyoxyl castor oil, nonylphenol ethoxylate cyclodextrins, lecithin, methylbenzethonium chloride, petroleum sulphonates, alkylbenzenesulphonates, sulphated alkanolamides, polyoxyethylene surfactants, carboxylic esters, polyethylene glycol esters, glycol esters of fatty acids, carboxylic amides, quaternary ammonium salts, preferably docusate sodium and/or sodium lauryl sulfate.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said one or more pharmaceutically excipients are selected from group consisting of: diluents or fillers, binders, disintegrants and dispersing agents, lubricants, glidants, antiadherents, surfactants, sweeteners and flavourings, and wherein said sweeteners are selected from: sodium saccharine, sucrose, saccharine, aspartame, acesulfame-K, sodium cyclamate and sorbitol.

In another embodiment of the invention, said abiraterone acetate is present in said stable, amorphous matrix at an amount ranging from about 1.0% by weight to about 95.0% by weight, based on the total weight of the stable, amorphous matrix.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said stable, amorphous matrix is present at an amount ranging from about 1.0% by weight to about 95.0% by weight, based on the total weight of the solid dosage form, preferably wherein said stable, amorphous matrix is present in particulate form, preferably as a particulate or milled extrudate.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said solid dosage form is selected from: a tablet, coated tablet, effervescent tablet, tablet for oral suspension, fast melt tablet, lyophilized tablet, lyophilized wafer, disintegrating tablet, dispersible tablet, orodispersible tablet, mini-tablet, multilayer tablet, bi-layered tablet, tablet-in-tablet, pill, micro-pellet, small tablet unit, powder, powder for reconstitution, powder for oral suspension, pellets, beads, UPS (multiple unit pellet system), granules, dry granules, roller compacted granules, effervescent granules, granules for oral suspension, slugs, microspheres, multiparticulates, sprinkles, preferably granules for oral suspension, wherein said solid dosage form is preferably packaged as a blister, bottle, capsule, stick or sachet.

In yet another embodiment of the invention, the micro-pellet, small tablet unit, powder, powder for reconstitution, powder for oral suspension, pellets, beads, MUPS (multiple unit pellet system), granules, dry granules, roller compacted granules, effervescent granules, granules for oral suspension, slugs, microspheres, multiparticulates and sprinkles preferably have a D50 of greater than 300 micron and where less than about 10% by weight of the total mass of the solid dosage form is less than 63 microns, or preferably wherein less than about 10% by weight of the total mass of the solid dosage form is less than 50 microns, as measured by laser diffraction or preferably sieve fractionation.

Alternatively, the micro-pellet, small tablet unit, powder, powder for reconstitution, powder for oral suspension, pellets, beads, MUPS (multiple unit pellet system), granules, dry granules, roller compacted granules, effervescent granules, granules for oral suspension, slugs, microspheres, multiparticulates and sprinkles have a particle size such that about 70% or about 80% or 90% or more are retained on a 270 mesh screen (53 microns), or where about 70% to about 80% or 90% or more are retained on a 230 mesh screen (63 microns), or where about 60% or about 70% or more are retained on a 60 mesh screen (250 microns), or where about 50% or more are retained on a 48 mesh screen (300 microns).

In yet another embodiment of the invention, the micro-pellet, small tablet unit, powder, powder for reconstitution, powder for oral suspension, pellets, beads, MUPS (multiple unit pellet system), granules, dry granules, roller compacted granules, effervescent granules, granules for oral suspension, slugs, microspheres, multiparticulates and sprinkles preferably have a bulk density of about 0.3 to about 0.95 g/ml. or about 0.4 to about 0.90 g/ml or about 0.7 to about 0.85 g/ml.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said solid dosage form is presented as one of the following: controlled release formulation, immediate release formulation, fast melt formulation, lyophilized formulation, delayed release formulation, extended release formulation, pulsatile release formulation, or mixed immediate release and controlled release formulation.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said solid dosage form for oral administration comprises a therapeutically effective amount of abiraterone acetate, ranging from about 10 mg to 2000 mg per dosage form, or about 50 mg to 1000 mg per dosage form, preferably about 62.5 mg to about 250 mg per dosage form.

In some embodiments, a therapeutically-effective amount is an amount sufficient to prevent or delay tumour or cancer recurrence. A therapeutically-effective amount can be administered in one or more administrations. The therapeutically-effective amount of abiraterone acetate, may result in one or more of the following: (i) reduce the number of cancer cells; (ii) reduce tumour size; (iii) inhibit, retard, slow to some extent and preferably stop cancer cell infiltration into peripheral organs; (iv) inhibit (i.e., slow to some extent and preferably stop) tumour metastasis; (v) inhibit tumour growth; (vi) prevent or delay occurrence and/or recurrence of tumour; and/or (vii) relieve to some extent one or more of the symptoms associated with the cancer. For example, for the treatment of tumours, a “therapeutically effective dosage” may induce tumour shrinkage by at least about 5% relative to baseline measurement, such as at least about 10%, or about 20%, or about 60% or more. The baseline measurement may be derived from untreated subjects. A therapeutically-effective amount of a therapeutic compound can decrease tumour size, or otherwise ameliorate symptoms in a subject. One of ordinary skill in the art would be able to determine such amounts based on such factors as the subject's size, the severity of the subject's symptoms, and the particular composition or route of administration selected. The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells.

The terms “effective amount” or “pharmaceutically effective amount” “or therapeutically effective amount”, refer to a sufficient amount of abiraterone acetate to provide the desired biological or therapeutic result. That result can be reduction, amelioration, palliation, lessening, delaying, and/or alleviation of one or more of the signs, symptoms, or causes of a cancer, preferably prostate cancer, or any other desired alteration of a biological system. In reference to prostate cancer, an effective amount may comprise an amount sufficient to cause a tumour to shrink and/or to decrease the growth rate of the tumour (such as to suppress tumour growth) or to prevent or delay other unwanted cell proliferation. In some embodiments, an effective amount is an amount sufficient to delay development, or prolong survival or induce stabilisation of the cancer or tumour. Preferably, therapeutic efficacy is measured by a decrease or stabilisation of tumour size of one or more said tumours and/or metastases, as defined by RECIST 1.1, including stable diseases (SD), a complete response (CR) or partial response (PR) of the target tumour; and/or stable disease (SD) or complete response (CR) of one or more non-target tumours. Alternatively, therapeutic efficacy is assessed by Immune Related Response Criteria (irRC), iRECIST or irRECIST, as would be known to the skilled person.

In another embodiment of the invention is provided a solid dosage form for oral administration which disintegrates and/or releases abiraterone acetate into an aliquot of liquid suitable for human consumption.

In yet another embodiment of the invention is provided a solid dosage form for oral administration which disintegrates and/or releases abiraterone acetate into an aliquot of liquid suitable for human consumption, wherein said aliquot of liquid suitable for human use is selected from: water, fruit juice such as orange juice, apple juice, pineapple juice or cranberry juice, coconut water, coconut milk, almond milk, oat milk, soya milk, rice milk, and dairy-derived milks such as cow's milk.

In another embodiment of the invention the solid dosage form for oral administration disintegrates and/or releases at least 85% or more of said abiraterone acetate into an aliquot of liquid in less than about 10 minutes, preferably less than about 5 minutes, more preferably less than about 3 minutes, or less than about 1 minute or less, wherein said aliquot is 250 ml or less, such as about 50 ml or about 60 ml, or between about 50 ml to about 100 ml, or other such small volume as required or preferred by the human patient.

In another embodiment of the invention the solid dosage form for oral administration disintegrates and/or releases at least 85% or more of said abiraterone acetate into an aliquot of liquid in less than about 10 minutes, preferably less than about 5 minutes, more preferably less than about 3 minutes, or less than about 1 minute or less, wherein said aliquot is 250 ml or less and results in a homogeneous suspension, wherein said formed suspension has a smooth consistency and does not contain a significant presence of caked or clumping particles or lumps, by visual inspection.

In another embodiment of the invention is provided a solid dosage form for oral administration wherein said solid dosage form for oral administration is capable of rapid reconstitution or disintegration in the oral cavity or an aliquot of liquid prior to ingestion such that patients with dysphagia can take it with ease.

In another embodiment of the invention is provided a method of treating, reducing, inhibiting or controlling prostate cancer in a human patient in need thereof, who is also suffering from dysphagia, comprising administering a therapeutically effective amount of a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion by said human patient.

In another embodiment of the invention is provided a solid dosage form for oral administration wherein said solid dosage form for oral administration is capable of rapid reconstitution or disintegration in the oral cavity or an aliquot of liquid prior to ingestion, such as sachets for oral suspension, powders to be swallowed, tablets to be chewed, tablets to be swallowed, tablets to be sucked, orodispersible tablets or water-dispersible tablets and such like.

In another embodiment of the invention is provided a solid dosage form for oral administration wherein said solid dosage form for oral administration is capable of rapid reconstitution or disintegration in the oral cavity or an aliquot of liquid prior to ingestion, which consists of a solid, porous, rapidly disintegrating solid oral dosage form.

In another embodiment of the invention is provided a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said abiraterone acetate is present in said solid dosage form at about 1.0% by weight to about 20.0% by weight, or about 1.0% to about 10.0% by weight, preferably about 1.0% to about 5.0% by weight, based on the total weight of the solid dosage form.

In a further embodiment of the invention is provided, a solid dosage form for oral administration comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, comprising:
(a) 1-20% abiraterone acetate,
(b) 5-60% water soluble polymer,
(c) 35-90% fillers and/or diluents,
(d) 5-20% binders,
(e) 5-45% disintegrants,
(f) 0.1-5% lubricants,
(g) 0.1-5% antiadherents,
(h) 0.1-5% glidants,
(i) 0.01-2% antioxidants, and
(j) 0.05-5% flavouring agents and/or sweeteners.

In a preferred embodiment of the invention is provided, a solid dosage form for oral administration comprising: (a) abiraterone acetate; (b) one or more water soluble polymers, and (c) one or more antioxidants, wherein said solid oral dosage form further comprises a disintegrant and one or more pharmaceutically acceptable excipients, and wherein said solid dosage form for oral administration is capable of rapid reconstitution or disintegration in the oral cavity or an aliquot of liquid prior to ingestion, for use in the treatment of prostate cancer.

In a further preferred embodiment of the invention is the use of a solid dosage form for oral administration according to the invention disclosed herein, in the manufacture of a medicament for the treatment of prostate cancer.

In another preferred embodiment of the invention is a method of producing a solid dosage form for oral administration comprising a stable, amorphous matrix comprising: (a) abiraterone acetate; (b) one or more water soluble polymers, and (c) one or more antioxidants, wherein said solid oral dosage form further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said method comprising the steps of:
(a) providing abiraterone acetate;
(b) providing one or more water-soluble polymers, preferably polyvinylpyrrolidone, such as K12 or Kollidon 17PF, Kollidon 25, Kollidon 30 or Kollidon 90, most preferably a K12 grade;
(c) providing one or more antioxidants;
(d) processing (a), (b) and (c) by blending to produce powder blend comprising (a) to (c);
(e) processing the product of step (d) by an extrusion process to produce an extruded composition;
(f) processing the extruded composition of step (e) by size reduction such as milling, to produce a particulate or milled extrudate;
(g) blending the particulate or milled extruded composition of step (f) with one or more pharmaceutically acceptable excipients such as fillers, diluents, binders, disintegrants, lubricants, antiadherents, glidants, flavoring agents and sweeteners to produce a composition blend; and
(h) compressing the composition blend of step (g) into a solid dosage form such as via direct compression to form a tablet, or, preparing granules from the composition blend of step (g), via slugging and/or milling and/or roller compaction to produce such granules.

In another preferred embodiment of the invention is a method of producing a solid dosage form for oral administration comprising a stable, amorphous matrix comprising: (a) abiraterone acetate; (b) one or more water soluble polymers, and (c) one or more antioxidants, wherein said solid oral dosage form further comprises a disintegrant and one or more pharmaceutically acceptable excipients, and wherein said solid dosage form for oral administration is capable of rapid reconstitution or disintegration in the oral cavity or an aliquot of liquid prior to ingestion, wherein said stable, amorphous complex is manufactured via an extrusion process, which preferably comprises a hot melt extrusion (HME) process.

In another embodiment of the invention is a method of treatment of prostate cancer comprising administering a therapeutically effective amount of a solid dosage form for oral administration according to any of the embodiments disclosed herein, to a human patient in need thereof.

A rapidly disintegrating solid oral dosage form of the invention can be prepared by lyophilizing said stable, amorphous matrix comprising: (a) abiraterone acetate; (b) one or more water soluble polymers, and (c) one or more antioxidants, together with one or more stabilizer and/or diluent. Suitable lyophilization conditions include, for example, those described in EP 0,363,365 (McNeil-PPC Inc.), U.S. Pat. No. 4,178,695 (A. Erbeia), and U.S. Pat. No. 5,384,124 (Farmalyoc), all of which are incorporated herein by reference. A preferred water-soluble carrier is pullulan, as described in WO2004/043440 (Elan Pharma International Ltd) or WO2000/0050013 (Quadrant Healthcare [UK] Ltd) each of which are incorporated by reference. Typically, the dispersion comprising said stable, amorphous matrix is placed in a suitable vessel and frozen to a temperature of between about −5° C. to about −100° C. The frozen dispersion is then subjected to reduced pressure for a period of up to about 48 hours. The combination of parameters such as temperature, pressure, dispersion medium, and batch size will impact the time required for the lyophilization process. Under conditions of reduced temperature and pressure, the frozen solvent is removed by sublimation yielding a solid, porous, rapidly disintegrating solid oral dosage form having the active ingredient distributed throughout.

In yet another embodiment of the invention is a solid dosage form for oral administration substantially as herein described with reference to the examples.

In a preferred embodiment of the invention is provided a method of treating, reducing, inhibiting or controlling prostate cancer in a human patient in need thereof, comprising administering a therapeutically effective amount of a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion by said human patient.

In a further embodiment of the method of the invention, said solid dosage form for oral administration is selected from: a tablet, coated tablet, effervescent tablet, tablet for oral suspension, fast melt tablet, lyophilized tablet, lyophilized wafer, disintegrating tablet, dispersible tablet, orodispersible tablet, mini-tablet, multilayer tablet, bilayered tablet, tablet-in-tablet, pill, micro-pellet, small tablet unit, powder, powder for reconstitution, powder for oral suspension, pellets, beads, MUPS (multiple unit pellet system), granules, dry granules, roller compacted granules, effervescent granules, granules for oral suspension, slugs, microspheres, multiparticulates, sprinkles, preferably granules for oral suspension, wherein said solid dosage form is preferably packaged as a blister, bottle, capsule, stick or sachet.

In a further embodiment of the method of the invention, said solid dosage form for oral administration is presented as one of the following: controlled release formulation, immediate release formulation, fast melt formulation, lyophilized formulation, delayed release formulation, extended release formulation, pulsatile release formulation, or mixed immediate release and controlled release formulation.

In a preferred embodiment of the invention is provided a method of treating, reducing, inhibiting or controlling prostate cancer in a human patient in need thereof, comprising administering a therapeutically effective amount of a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion by said human patient, wherein said administration results in one or more of the following features:
(a) a peak abiraterone plasma concentration which is about 248 ng/ml to 662 ng/ml and a more uniform abiraterone plasma concentration than the abiraterone plasma concentration provided by the same dosage of a conventional abiraterone acetate tablet formulation;
(b) an abiraterone exposure (AUC) which is about 500 h*ng/ml to 1350 h*ng/ml and a more uniform abiraterone exposure (AUC) than the abiraterone exposure (AUC) provided by the same dosage of a conventional abiraterone acetate tablet formulation, such as Zytiga;
(c) exhibits no positive food effect, such as where the fed/fasted ratio is under 1.25;
(d) allows significant dose reduction or dose sparing when compared to Zytiga;
(e) abandonment of the requirement for taking the drug on an empty stomach;
(f) the variability of exposure is significantly reduced when compared to Zytiga.

In a further embodiment of the invention is provided, a solid dosage form for oral administration comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, characterized in that it possesses at least one of the following material properties:
(i) is instantaneously redispersible in physiological relevant media;
(ii) has a disintegration time not more than 5 minutes, preferably not more than 3 minutes in a glass of water;
(iii) has increased dissolution rate in a glass of water—Q=80% at 15 minutes, preferably at 5 minutes;
(iv) shows X-ray amorphous character in the solid form;
(v) stable for at least 3 months at 40° C. without any significant decomposition and/or oxidation, and/or crystallization.

In a further embodiment, the solid dosage form preferably comprises a polyvinylcaprolactampolyvinyl acetate-polyethylene-glycol graft copolymer (Soluplus®), and sodium deoxcycholate. Soluplus proved to be effective in preventing the rapid aggregation and/or Ostwald ripening of the reconstituted/dispersed solid dosage form of the invention, particularly granules for oral suspension, and thus keeping the abiraterone acetate particles dispersed/keeping abiraterone acetate apparently dissolved ensuring the API availability for absorption. The bile salt sodium deoxycholate (SDC) was beneficial in obtaining redispersable solid forms and to prevent the rapid aggregation/growth of abiraterone acetate particles. In certain embodiments or dosage forms, Soluplus and SDC enabled the in-use stability of the reconstituted suspension to be at least 30% to 50% or more (where abiraterone content is measured by a filtration test).

In a further embodiment of the invention is provided a method of treating, reducing, inhibiting or controlling prostate cancer in a human patient in need thereof, comprising administering a therapeutically effective amount of a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion by said human patient, wherein the therapeutically effective amount of abiraterone acetate administered is from about 62.5 mg/day to about 1000 mg/day, preferably from about 62.5 mg/day to about 725 mg/day and more preferably from about 62.5 mg/day to about 500 mg/day, most preferably as a divided dose.

In an embodiment of the invention is provided a method of treating, reducing, inhibiting or controlling prostate cancer in a human patient in need thereof, comprising administering a therapeutically effective amount of a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion by said human patient, wherein the therapeutically effective amount of abiraterone acetate is administered as a total daily dose of between about 62.5 mg and 250 mg abiraterone acetate administered once a day, two times a day (BID), three times a day (TID), or four times a day (QID), optionally wherein said about 62.5 mg and 250 mg total dose comprises administration of between one to four 62.5 mg tablets or sachets suitably comprising granules for oral suspension.

Alternatively, the therapeutically effective amount of abiraterone acetate is administered as a total daily dose of between about 62.5 mg and 1000 mg abiraterone acetate administered in a single or divided dose, such as between about 62.5 mg and 1000 mg administered once a day, two times a day (BID), three times a day (TID), or four times a day (QID).

Alternatively, the therapeutically effective amount of abiraterone acetate is administered as a total daily dose of between about 62.5 mg and 1000 mg or between about 125 and 750 mg or about 250 mg and 500 mg abiraterone acetate administered in a single or divided dose, wherein said dose comprises administration of between one to four 62.5 mg sachets suitably comprising granules for oral suspension, administered once, twice, three or four times a day, such as four sachets containing 62.5 mg abiraterone acetate, administered twice a day, e.g. morning and night.

A further alternative includes where the therapeutically effective amount of abiraterone acetate is administered as a total daily dose of between about 62.5 mg and 1000 mg or between about 125 mg and 750 mg or between about 250 mg and 500 mg abiraterone acetate, administered in a single or divided dose, wherein said dose comprises administration of between one to four 62.5 mg sachets suitably comprising granules for oral suspension, administered once, twice, three or four times a day, such as four sachets containing 62.5 mg abiraterone acetate, administered twice a day, e.g. morning and night.

A further alternative includes where the therapeutically effective amount of abiraterone acetate is administered as a total daily dose of between about 62.5 mg and 1000 mg or between about 125 and 750 mg or between about 250 mg and 500 mg abiraterone acetate, administered in a single or divided dose of between one to four 250 mg tablets administered once, twice, three or four times a day.

A further embodiment includes where the therapeutically effective amount of abiraterone acetate is administered as a total dose of 500 mg abiraterone acetate administered wherein said 500 mg total dose comprises administration of four 62.5 mg sachets suitably comprising granules for oral suspension, twice daily.

In an embodiment where said human patient exhibits hepatic impairment, the therapeutically effective amount of abiraterone acetate is administered as a total dose of between 250 and about 370 mg abiraterone acetate, wherein said 250 mg total dose comprises administration of two 62.5 mg sachets suitably comprising granules for oral suspension, twice daily; or wherein said 375 mg total dose comprises administration of three 62.5 mg sachets suitably comprising granules for oral suspension, twice daily.

In a preferred embodiment of the invention is provided a method of treating, reducing, inhibiting or controlling prostate cancer in a human patient in need thereof, comprising administering a therapeutically effective amount of a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion by said human patient, further comprising simultaneously, separately or sequentially administering a therapeutically-effective amount of a steroid, wherein said steroid is selected from: hydrocortisone, hydrocortisone acetate, cortisone acetate, tixocortol pivalate, prednisolone, methylprednisolone, prednisone, or dexamethasone, preferably prednisone.

As used herein, the terms “concurrent administration” or “concurrently” or “simultaneous” mean that administration occurs on the same day. The terms “sequential administration” or “sequentially” or “separate” mean that administration occurs on different days.

“Simultaneous” administration, as defined herein, includes the administration of the solid dosage form for oral administration according to the invention and administering a therapeutically-effective amount of a steroid, within about 2 hours or about 1 hour or less of each other, even more preferably at the same time.

“Separate” administration, as defined herein, includes the administration of a solid dosage form for oral administration according to the invention and administering a therapeutically-effective amount of a steroid, more than about 12 hours, or about 8 hours, or 15 about 6 hours or about 4 hours or about 2 hours apart.

“Sequential” administration, as defined herein, includes the administration of the solid dosage form for oral administration according to the invention and administering a therapeutically-effective amount of a steroid, each in multiple aliquots and/or doses and/or on separate occasions.

In a further preferred embodiment of the invention is provided a method of treating, reducing, inhibiting or controlling prostate cancer in a human patient in need thereof, comprising administering a therapeutically effective amount of a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion by said human patient, wherein said prostate cancer in a human patient is selected from: high-risk Castrate Sensitive Prostate Cancer (CSPC), metastatic Castrate Sensitive Prostate Cancer (mCSPC) and metastatic Castrate Resistant Prostate Cancer (mCRPC).

In a further embodiment of the invention is provided a method of treating, reducing, inhibiting or controlling prostate cancer in a human patient in need thereof, comprising administering a therapeutically effective amount of a solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion by said human patient, wherein said therapeutically effective amount of a solid dosage form for oral administration is first introduced into an aliquot of liquid suitable for human consumption, prior to ingestion by said human patient, preferably wherein said aliquot is 250 ml or less, such as about 50 or 60 ml.

In a further embodiment of the invention, said aliquot of liquid suitable for human use is selected from: water, fruit juice such as orange juice, apple juice, pineapple juice or cranberry juice, coconut water, coconut milk, almond milk, oat milk, soya milk, rice milk, and dairy-derived milks such as cow's milk.

A method for the treatment of a prostate cancer in a human comprises administering an abiraterone acetate tablet for oral suspension or abiraterone acetate granules for oral suspension formulation comprising a therapeutically effective amount of abiraterone acetate and a therapeutically effective amount of steroid to produce
(a) a peak abiraterone plasma concentration which is about 248 ng/ml to 662 ng/ml and a more uniform abiraterone plasma concentration than the abiraterone plasma concentration provided by the same dosage of a conventional abiraterone acetate tablet formulation; and
(b) an abiraterone exposure (AUC) which is about 500 h*ng/ml to 1350 h*ng/ml and a more uniform abiraterone exposure (AUC) than the abiraterone exposure (AUC) provided by the same dosage of a conventional abiraterone acetate tablet formulation.

The method for the treatment of a prostate cancer, wherein the therapeutically effective amount of the abiraterone acetate is from about 62.5 mg/day to about 1000 mg/day.

The method for the treatment of a prostate cancer, wherein the therapeutically effective amount of the abiraterone acetate is from about 62.5 mg/day to about 725 mg/day.

The method for the treatment of a prostate cancer, wherein the therapeutically effective amount of the abiraterone acetate is from about 62.5 mg/day to about 500 mg/day, most preferably as a divided dose.

The method for the treatment of a prostate cancer, wherein said steroid is chosen from hydrocortisone, hydrocortisone acetate, cortisone acetate, tixocortol pivalate, prednisolone, methylprednisolone, prednisone and dexamethasone.

The method for the treatment of a prostate cancer, wherein the therapeutically effective amount of the abiraterone acetate is from about 62.5 mg/day to about 1000 mg/day, preferable from about 62.5 mg/day to about 725 mg/day and more preferable from about 62.5 mg/day to about 500 mg/day.

The method for the treatment of a prostate cancer, wherein the therapeutically effective amount of the abiraterone acetate in the form of tablet for oral suspension or granules for oral suspension is administered orally as one (1) of 250 mg abiraterone acetate tablets for oral suspension or one (1) of 250 mg sachets containing abiraterone acetate granules for oral suspension once a day or 2 times a day (BID) or 3 times a day (TID) or 4 times a day (QID).

The method for the treatment of a prostate cancer, wherein the therapeutically effective amount of the abiraterone acetate in the form of tablet for oral suspension or granules for oral suspension is administered orally as one (1) of 250 mg abiraterone acetate tablets for oral suspension or one (1) of 250 mg sachets containing abiraterone acetate granules for oral suspension 2 times a day (BID).

The method for the treatment of a prostate cancer, wherein the therapeutically effective amount of the abiraterone acetate in the form of tablet for oral suspension or granules for oral suspension is administered orally as one (1) to four (4) of 62.5 mg abiraterone acetate tablets for oral suspension or one (1) to four (4) of 62.5 mg sachets containing abiraterone acetate granules for oral suspension once a day or 2 times a day (BID) or 3 times a day (TID) or 4 times a day (QID).

The method for the treatment of a prostate cancer, wherein the therapeutically effective amount of the abiraterone acetate in the form of tablet for oral suspension or granules for oral suspension is administered orally as one (1) to four (3) of 62.5 mg abiraterone acetate tablets for oral suspension or one (1) to four (3) of 62.5 mg sachets containing abiraterone acetate granules for oral suspension once a day.

The method for the treatment of a prostate cancer, wherein said prostate cancer in a human is a high-risk castrate sensitive prostate cancer (CSPC) and metastatic castrate resistant prostate cancer (mCRPC).

An abiraterone acetate tablet for oral suspension or abiraterone acetate granules for oral suspension comprising
(a) abiraterone acetate as active ingredient;
(b) water soluble polymer matrix selected from polyvinylcaprolactam-polyvinyl acetate-polyethylene-glycol graft copolymers, poloxamers; polyvinylpyrrolidone, poly(acrylic acid), polyvinyl alcohol, ethylene glycol and vinyl alcohol graft copolymer, hydroxypropyl methylcellulose, hydroxypropylcellulose, hydroxyethylcellulose, hypromellose acetate succinate, polyethylene oxide, polyethylene-glycol, poly(2-Ethyl-2-oxazoline), poly(methyl vinyl ether/maleic anhydride), poly(maleic acid-co-methyl vinyl ether, polyoxylglycerides, D-α-tocopherol polyethylene glycol 1000 succinate, polyoxyethylene castor oil derivatives, polyoxyethylene sorbitan fatty acid esters, polylactic acid, poly(lactic-co-glycolic acid) and copolymers of vinylpyrrolidone and vinyl-acetate and poly(maleic acid-co-methyl-vinyl-ether);
(c) fillers and/or diluents chosen from lactose (anhydrous), lactose monohydrate, mannitol, sucrose, glucose, plant cellulose, calcium carbonate, magnesium carbonate, magnesium oxide, microcrystalline cellulose, silicified microcrystalline cellulose, sorbitol, starch, pregelatinized starch, isomalt, anhydrous dibasic calcium phosphate, dibasic calcium phosphate dihydrate, calcium silicate, magnesium aluminium silicate, maltodextrin, dextrates;
(d) binders selected from cellulose, methyl cellulose, microcrystalline cellulose, cellulose ethers such as hydroxypropyl cellulose, polyvinylpyrrolidone, polyethylene glycol, lactose, sucrose, mannitol, sorbitol and xylitol and their co-processed versions such as alpha-Lactose-monohydrate and cellulose or starch, urea crystals, sodium sulfate and calcium sulfate dihydrate;
(e) disintegrants and dispersing agents selected from polyvinylpyrrolidone, crosslinked polyvinylpyrrolidone (crospovidone), polyvinylcaprolactam-polyvinyl acetate-polyethylene-glycol graft copolymers, carboxymethyl cellulose, crosslinked cellulose and its sodium salt (crosscarmellose), crosslinked sodium carboxymethyl cellulose, modified starch, sodium starch glycolate, crosslinked starch, crosslinked alginic acid and sodium starch glycolate, colloidal silicon dioxide, soy polysaccharides and sodium deoxycholate;
(f) lubricants chosen from polyethylene glycol, magnesium stearate, silica, talc, stearic acid, sodium stearyl fumarate, sodium lauryl sulfate, sodium benzoate, stearic acid and magnesium stearate;
(g) antiadherents: magnesium stearate, talc and starch;
(h) glidants selected from silica gel, colloidal silicon dioxide, fumed silica, talc and magnesium carbonate;
(i) antioxidants selected from ascorbic acid, ascorbyl palmitate, butylated hydroxyanisole, butylated hydroxytoluene, hypophosphorous acid, monothioglycerol, potassium metabisulfite, propyl gallate, sodium bisulfite, sodium formaldehyde sulfoxylate, sodium metabisulfite, sodium sulfite, sodium thiosulfate and tocopherols; and
(j) flavoring agents and sweeteners chosen from sodium saccharine, sucrose, saccharine, aspartame, acesulfame-K, sodium cyclamate and sorbitol.

The abiraterone acetate tablet for oral suspension or abiraterone acetate granules for oral suspension comprising
(a) 1-20% abiraterone acetate as active ingredient;
(b) 5-60% water soluble polymer matrix selected from polyvinylcaprolactam-polyvinyl acetate-polyethylene-glycol graft copolymers, poloxamers; polyvinylpyrrolidone, poly(acrylic acid), polyvinyl alcohol, ethylene glycol and vinyl alcohol graft copolymer, hydroxypropyl methylcellulose, hydroxypropylcellulose, hydroxyethylcellulose, hypromellose acetate succinate, polyethylene oxide, polyethylene-glycol, poly(2-Ethyl-2-oxazoline), poly(methyl vinyl ether/maleic anhydride), poly(maleic acid-co-methyl vinyl ether, polyoxylglycerides, D-α-tocopherol polyethylene glycol 1000 succinate, polyoxyethylene castor oil derivatives, polyoxyethylene sorbitan fatty acid esters, polylactic acid, poly(lactic-co-glycolic acid) and copolymers of vinylpyrrolidone and vinyl-acetate and poly(maleic acid-co-methyl-vinyl-ether), copolymers of vinylpyrrolidone and vinyl-acetate, and poly(maleic acid-co-methyl-vinyl-ether);
(c) 35-90% fillers and/or diluents chosen from lactose (anhydrous), lactose monohydrate, mannitol, sucrose, magnesium stearate, glucose, plant cellulose, calcium carbonate; magnesium carbonate, magnesium oxide, microcrystalline cellulose, sorbitol, and starch;
(d) 5-20% binders selected from cellulose, methyl cellulose, microcrystalline cellulose, cellulose ethers such as hydroxypropyl cellulose, polyvinylpyrrolidone, polyethylene glycol, lactose, sucrose, microcrystalline cellulose, mannitol, sorbitol, and xylitol;
(e) 5-45% disintegrants and dispersing agents selected from polyvinylpyrrolidone, crosslinked polyvinylpyrrolidone (crospovidone), polyvinylcaprolactam-polyvinyl acetate-polyethylene-glycol graft copolymers, carboxymethyl cellulose, crosslinked cellulose and its sodium salt (crosscarmellose), crosslinked sodium carboxymethyl cellulose, modified starch sodium starch glycolate, crosslinked starch, crosslinked alginic acid and sodium starch glycolate, and sodium deoxycholate;
(f) 0.1-5% lubricants chosen from polyethylene glycol, magnesium stearate, silica, talc, stearic acid, sodium stearyl fumarate, and magnesium stearate;
(g) 0.1-5% antiadherents: magnesium stearate, talc, and starch;
(h) 0.1-5% glidants selected from silica gel, colloidal silicon dioxide, fumed silica, talc, and magnesium carbonate;
(i) 0.01-2% antioxidants selected from ascorbic acid, ascorbyl palmitate, butylated hydroxyanisole, butylated hydroxytoluene, hypophosphorous acid, monothioglycerol, potassium metabisulfite, propyl gallate, sodium bisulfite, sodium formaldehyde sulfoxylate, sodium metabisulfite, sodium sulfite, sodium thiosulfate, and tocopherols;
(j) 0.05-5% flavoring agents and sweeteners chosen from sodium saccharine, sucrose, saccharine, aspartame, acesulfame-K, sodium cyclamate, and sorbitol.

The abiraterone acetate tablet for oral suspension or abiraterone acetate granules for oral suspension, wherein comprises from about 1 to about 20% abiraterone acetate, preferable from about 1 to about 15% abiraterone acetate, more preferable from about 1 to about 5% abiraterone acetate.

The abiraterone acetate tablet for oral suspension or abiraterone acetate granules for oral suspension, wherein said tablet for oral suspension or granules for oral suspension is suitable for oral administration.

The abiraterone acetate tablet for oral suspension or abiraterone acetate granules for oral suspension, wherein tablet for oral suspension or granules for oral suspension disintegrates and release abiraterone acetate in liquids suitable for human consumption.

The abiraterone acetate tablet for oral suspension or abiraterone acetate granules for oral suspension disintegrates and release abiraterone acetate in liquids, wherein said liquids suitable for human use selected from water, orange juice, apple juice, pineapple juice, cranberry juice and milk.

The abiraterone acetate tablet for oral suspension or abiraterone acetate granules for oral suspension, wherein said tablet for oral suspension or granules for oral suspension disintegrates and release abiraterone acetate in liquids in less than about 10 minutes, preferable less than about 5 minutes, more preferably less than about 3 minutes or less than about 1 minute or less, in liquids suitable for human consumption.

A method of producing a abiraterone acetate tablet for oral suspension or abiraterone acetate granules for oral suspension composition comprising abiraterone acetate, wherein said method comprising the steps of:
(a) providing abiraterone acetate;
(b) providing a water soluble polymer matrix;
(c) providing one or more antioxidants;
(d) processing (a), (b) and (c) by blending to produce powder blend comprising (a) to (c);
(e) processing the product of step (d) by an extrusion process to produce an extruded composition;
(f) processing the extruded composition by milling to produce milled extrudate;
(g) blending the milled extruded composition with one or more pharmaceutically acceptable excipients such as fillers, diluents, binders, disintegrants, dispersing agents, lubricants, antiadherents, glidants, flavoring agents and sweeteners to produce a composition blend; and
(h) directly compressing the composition blend into a direct compression tablet or preparing granules with slugging and milling or preparing granules using roller compaction.

In one embodiment, the extrusion process is a hot melt extrusion (MIE) process and the roller compaction process utilizes a suitable sieve size, compaction force, gap size, press roller start speed, press roller speed and granulator speed as would be known to the skilled person.

The present invention further provides an abiraterone acetate tablet for oral suspension or abiraterone acetate granules for oral suspension compositions and methods for the treatment of high-risk Castrate Sensitive Prostate Cancer (CSPC), metastatic Castrate Sensitive Prostate Cancer (mCSPC) and metastatic Castrate Resistant Prostate Cancer (mCRPC).

The present invention further relates to a novel abiraterone acetate tablet for oral suspension or abiraterone acetate granules for oral suspension characterized by improved physicochemical properties comprising
(a) abiraterone acetate hot-melt extrudate intra-granular matrix comprising abiraterone acetate as active compound and pharmaceutical excipients chosen from polyvinylcaprolactam-polyvinyl acetate-polyethylene-glycol graft copolymers; polyvinylpyrrolidone; copolymers of vinylpyrrolidone and vinyl-acetate; and
(b) an extra-granular matrix comprising the mixture of pharmaceutical excipients selected from the group of co-processed versions of alpha-Lactose-monohydrate and cellulose or starch, anhydrous dibasic calcium phosphate, dibasic calcium phosphate dihydrate, crosslinked cellulose and its sodium salt (crosscarmellose), crosslinked sodium carboxymethyl cellulose, modified starch, sodium starch glycolate, magnesium stearate, sodium deoxycholate, polyvinylcaprolactam-polyvinyl acetate-polyethylene-glycol graft copolymers (Soluplus®), colloidal silicone dioxide, lactose monohydrate, microcrystalline cellulose, crospovidone, sodium saccharine dehydrate, polyethylene glycol and sodium stearyl fumarate;
   wherein said abiraterone acetate tablet for oral suspension or abiraterone acetate granules for oral suspension characterized in that it possesses at least one of the following material properties:
(a) is instantaneously redispersible in physiological relevant media;
(b) has a disintegration time not more than 5 minutes, preferably not more than 3 minutes in a glass of water;
(c) has increased dissolution rate in a glass of water—Q=80% at 15 minutes, preferably at 5 minutes;
(d) shows X-ray amorphous character in the solid form;
(e) stable for at least 3 months at 40° C. without any significant decomposition and/or oxidation, and/or crystallization.

In particular, non-limiting, embodiments of the invention, the therapeutically effective amount of abiraterone acetate is from about 250 mg/day to about 500 mg/day, most preferably as a divided dose, providing peak abiraterone plasma concertation (Cₘₐₓ) which is
(a) about 27 to about 55 ng/mL administering 62.5 mg therapeutically effective amount of abiraterone acetate orally in fasted state;
(b) about 69 to about 195 ng/mL administering 125 mg therapeutically effective amount of abiraterone acetate orally in fasted state;
(c) about 129 to about 392 ng/mL administering 187.5 mg therapeutically effective amount of abiraterone acetate orally in fasted state;
(d) about 248 to about 662 ng/mL administering 250 mg therapeutically effective amount of abiraterone acetate orally in fasted state;
(e) about 61 to about 190 ng/mL administering 250 mg therapeutically effective amount of abiraterone acetate orally in fed state; and
   exposure (AUC) which is
(a) about 53 to about 144 h*ng/mL administering 62.5 mg therapeutically effective amount of abiraterone acetate administered orally in fasted state;
(b) about 158 to about 409 h*ng/mL administering 125 mg therapeutically effective amount of abiraterone acetate orally in fasted state;
(c) about 314 to about 875 h*ng/mL administering 187.5 mg therapeutically effective amount of abiraterone acetate orally in fasted state;
(d) about 500 to about 1350 h*ng/mL administering 250 mg therapeutically effective amount of abiraterone acetate orally in fasted state;
(e) about 266 to about 761 h*ng/mL administering 250 mg therapeutically effective amount of abiraterone acetate orally in fed state; and
   significantly reduced variability of exposure when compared to commercially available formulations.

We have found that only the selected combinations of pharmaceutical excipients disclosed in the present invention result in a stable abiraterone acetate tablet for oral suspension or abiraterone acetate granules for oral suspension composition containing abiraterone acetate having improved physicochemical characteristics and enhanced biological performance.

Disclosed herein is a stable abiraterone acetate tablet for oral suspension or abiraterone acetate granules for oral suspension composition comprising
(a) abiraterone acetate as active ingredient;
(b) water soluble polymer matrix selected from polyvinylcaprolactam-polyvinyl acetate-polyethylene-glycol graft copolymers, poloxamers; polyvinylpyrrolidone, poly(acrylic acid), polyvinyl alcohol, ethylene glycol and vinyl alcohol graft copolymer, hydroxypropyl methylcellulose, hydroxypropylcellulose, hydroxyethylcellulose, hypromellose acetate succinate, polyethylene oxide, polyethylene-glycol, poly(2-Ethyl-2-oxazoline), poly(methyl vinyl ether/maleic anhydride), poly(maleic acid-co-methyl vinyl ether, polyoxylglycerides, D-α-tocopherol polyethylene glycol 1000 succinate, polyoxyethylene castor oil derivatives, polyoxyethylene sorbitan fatty acid esters, polylactic acid, poly(lactic-co-glycolic acid) and copolymers of vinylpyrrolidone and vinyl-acetate and poly(maleic acid-co-methyl-vinyl-ether);
(c) fillers and/or diluents chosen from lactose (anhydrous), lactose monohydrate, mannitol, sucrose, glucose, plant cellulose, calcium carbonate, magnesium carbonate, magnesium oxide, microcrystalline cellulose, silicified microcrystalline cellulose, sorbitol, starch, pregelatinized starch, isomalt, anhydrous dibasic calcium phosphate, dibasic calcium phosphate dihydrate, calcium silicate, magnesium aluminium silicate, maltodextrin, dextrates;
(d) binders selected from cellulose, methyl cellulose, microcrystalline cellulose, cellulose ethers such as hydroxypropyl cellulose, polyvinylpyrrolidone, polyethylene glycol, lactose, sucrose, mannitol, sorbitol and xylitol and their co-processed versions such as alpha-Lactose-monohydrate and cellulose or starch, urea crystals, sodium sulfate and calcium sulfate dihydrate;
(e) disintegrants and dispersing agents selected from polyvinylpyrrolidone, crosslinked polyvinylpyrrolidone (crospovidone), polyvinylcaprolactam-polyvinyl acetate-polyethylene-glycol graft copolymers, carboxymethyl cellulose, crosslinked cellulose and its sodium salt (crosscarmellose), crosslinked sodium carboxymethyl cellulose, modified starch, sodium starch glycolate, crosslinked starch, crosslinked alginic acid and sodium starch glycolate, colloidal silicon dioxide, soy polysaccharides and sodium deoxycholate;
(f) lubricants chosen from polyethylene glycol, magnesium stearate, silica, talc, stearic acid, sodium stearyl fumarate, sodium lauryl sulfate, sodium benzoate, stearic acid and magnesium stearate;
(g) antiadherents: magnesium stearate, talc and starch;
(h) glidants selected from silica gel, colloidal silicon dioxide, fumed silica, talc and magnesium carbonate;
(i) antioxidants selected from ascorbic acid, ascorbyl palmitate, butylated hydroxyanisole, butylated hydroxytoluene, hypophosphorous acid, monothioglycerol, potassium metabisulfite, propyl gallate, sodium bisulfite, sodium formaldehyde sulfoxylate, sodium metabisulfite, sodium sulfite, sodium thiosulfate and tocopherols;
(j) flavoring agents and sweeteners chosen from sodium saccharine, sucrose, saccharine, aspartame, acesulfame-K, sodium cyclamate and sorbitol.

In an embodiment, said water soluble polymer matrix is polyvinylpyrrolidone and/or copolymers of vinylpyrrolidone and vinyl-acetate.

In a preferred embodiment, said fillers and/or diluents are lactose (anhydrous), lactose monohydrate, co-processed versions of alpha-Lactose-monohydrate and cellulose or starch and microcrystalline cellulose.

In a preferred embodiment, said binders selected from cellulose, methyl cellulose, microcrystalline cellulose, cellulose ethers such as hydroxypropyl cellulose, polyvinylpyrrolidone, polyethylene glycol, lactose, microcrystalline cellulose.

In a preferred embodiment, said disintegrants and dispersing agents are crosslinked polyvinylpyrrolidone (crospovidone), polyvinylcaprolactam-polyvinyl acetate-polyethylene-glycol graft copolymers, sodium deoxycholate, crosslinked cellulose, sodium starch glycolate and polyvinylpyrrolidone.

In a preferred embodiment, said lubricants are polyethylene glycol, sodium stearyl fumarate and magnesium stearate.

In a preferred embodiment, said antiadherents: magnesium stearate, talc and starch.

In a preferred embodiment, said glidant is colloidal silicon dioxide.

In a preferred embodiment, said antioxidants are butylated hydroxyanisole and butylated hydroxytoluene.

In a preferred embodiment, said flavoring and sweetener is sodium saccharine and aspartame.

Further disclosed herein is a method of producing an abiraterone acetate tablet for oral suspension or abiraterone acetate granules for oral suspension composition comprising abiraterone acetate, said method comprising the steps of:
(a) providing abiraterone acetate;
(b) providing a water-soluble polymer matrix;
(c) providing one or more antioxidants;
(d) processing (a), (b) and (c) by blending to produce powder blend comprising (a) to (c);
(e) processing the product of step (d) by an extrusion process to produce an extruded composition;
(f) processing the extruded composition by milling to produce milled extrudate;
(g) blending the milled extruded composition with one or more pharmaceutically acceptable excipients such as fillers, diluents, binders, disintegrants, dispersing agents, lubricants, antiadherents, glidants, flavoring agents and sweeteners to produce a composition blend; and
(h) directly compressing the composition blend into a direct compression tablet or preparing granules with slugging and milling or preparing granules using roller compaction.

In an embodiment, said water soluble polymer matrix is polyvinylpyrrolidone or copolymers of vinylpyrrolidone and vinyl-acetate.

In a preferred embodiment, said antioxidants are hydroxyanisole and butylated hydroxytoluene.

In one embodiment, the extrusion process is a hot melt extrusion (HME) process.

In a preferred embodiment, said fillers and/or diluents are lactose (anhydrous), lactose monohydrate and microcrystalline cellulose; said binders selected from cellulose, methyl cellulose, microcrystalline cellulose, cellulose ethers such as hydroxypropyl cellulose, polyvinylpyrrolidone, polyethylene glycol, lactose, sucrose, microcrystalline cellulose, mannitol, sorbitol and xylitol; said disintegrants and dispersing agents are crosslinked polyvinylpyrrolidone (crospovidone), polyvinylcaprolactam-polyvinyl acetate-polyethylene-glycol graft copolymers and sodium deoxycholate; said lubricants are polyethylene glycol and sodium stearyl fumarate; said antiadherents: magnesium stearate, talc and starch; said glidant is colloidal silicon dioxide; said flavoring and sweetener is sodium saccharine.

In one embodiment, said tablet is an abiraterone acetate tablet for oral suspension or abiraterone acetate granules for oral suspension.

In one embodiment, said abiraterone acetate tablet for oral suspension or abiraterone acetate granules for oral suspension comprises from about 1 to about 20% abiraterone acetate, preferable from about 1 to about 15% abiraterone acetate, more preferable from about 1 to about 5% abiraterone acetate.

In an embodiment, said abiraterone acetate tablet for oral suspension or abiraterone acetate granules for oral suspension is suitable for oral administration.

In one embodiment, said abiraterone acetate tablet for oral suspension or abiraterone acetate granules for oral suspension disintegrates and release abiraterone acetate in liquids suitable for human consumption.

In one embodiment, said liquids suitable for human use selected from water, orange juice, apple juice, pineapple juice, cranberry juice and milk.

In one embodiment, said abiraterone acetate tablet for oral suspension or abiraterone acetate granules for oral suspension disintegrates and release abiraterone acetate in less than about 10 minutes, preferable less than about 5 minutes, more preferable less than about 3 minutes in liquids suitable for human consumption.

Dissolution rate of abiraterone acetate from tablet as well as granules in a glass of water is 85% at 15 minutes, preferably at 5 minutes.

Abiraterone acetate is X-ray amorphous in the solid form.

Further disclosed herein is a stable abiraterone acetate tablet for oral suspension or abiraterone acetate granules for oral suspension comprising
(a) 1-20% abiraterone acetate as active ingredient;
(b) 10-40% water soluble polymer matrix selected from polyvinylcaprolactam-polyvinyl acetate-polyethylene-glycol graft copolymers, poloxamers; polyvinylpyrrolidone, copolymers of vinylpyrrolidone and vinyl-acetate and poly(maleic acid-co-methyl-vinyl-ether);
(c) 35-60% fillers and/or diluents chosen from lactose (anhydrous), lactose monohydrate, mannitol, sucrose, magnesium stearate, glucose, plant cellulose, calcium carbonate; magnesium carbonate, magnesium oxide, microcrystalline cellulose, sorbitol and starch;
(d) 5-15% binders selected from cellulose, methyl cellulose, microcrystalline cellulose, cellulose ethers such as hydroxypropyl cellulose, polyvinylpyrrolidone, polyethylene glycol, lactose, sucrose, microcrystalline cellulose, mannitol, sorbitol and xylitol;
(e) 15-45% disintegrants and dispersing agents selected from polyvinylpyrrolidone, crosslinked polyvinylpyrrolidone (crospovidone), polyvinylcaprolactam-polyvinyl acetate-polyethylene-glycol graft copolymers, carboxymethyl cellulose, crosslinked cellulose and its sodium salt (crosscarmellose), crosslinked sodium carboxymethyl cellulose, modified starch sodium starch glycolate, crosslinked starch, crosslinked alginic acid and sodium starch glycolate and sodium deoxycholate;
(f) 0.1-5% lubricants chosen from polyethylene glycol, magnesium stearate, silica, talc, stearic acid, sodium stearyl fumarate and magnesium stearate;
(g) 0.1-3% antiadherents: magnesium stearate, talc and starch;
(h) 0.1-2% glidants selected from silica gel, colloidal silicon dioxide, fumed silica, talc and magnesium carbonate;
(i) 0.01-0.5% antioxidants selected from ascorbic acid, ascorbyl palmitate, butylated hydroxyanisole, butylated hydroxytoluene, hypophosphorous acid, monothioglycerol, potassium metabisulfite, propyl gallate, sodium bisulfite, sodium formaldehyde sulfoxylate, sodium metabisulfite, sodium sulfite, sodium thiosulfate and tocopherols;
(j) 0.05-1% flavoring agents and sweeteners chosen from sodium saccharine, sucrose, aspartame and sorbitol.

In an embodiment, said abiraterone acetate tablet for oral suspension or abiraterone acetate granules for oral suspension compositions is for use in the manufacture of a medicament for the treatment of a certain type of prostate cancer that has spread to other parts of the body and earlier stages of prostate cancer.

In an embodiment, said abiraterone acetate tablet for oral suspension or abiraterone acetate granules for oral suspension compositions is used for treatment of a certain type of prostate cancer that has spread to other parts of the body and earlier stages of prostate cancer.

In an embodiment, said certain type of prostate cancer is a high-risk Castrate Sensitive Prostate Cancer (CSPC), metastatic Castrate Sensitive Prostate Cancer (mCSPC) and metastatic Castrate Resistant Prostate Cancer (mCRPC).

In an embodiment, the administration of said abiraterone acetate tablet for oral suspension or abiraterone acetate granules for oral suspension provides peak abiraterone plasma concertation (Cₘₐₓ) which is about 27 to about 55 ng/mL when 62.5 mg therapeutically effective amount of abiraterone acetate administered orally in fasted state.

In an embodiment, the administration of said abiraterone acetate tablet for oral suspension or abiraterone acetate granules for oral suspension provides peak abiraterone plasma concertation (Cₘₐₓ) which is about 69 to about 195 ng/mL when 125 mg therapeutically effective amount of abiraterone acetate administered orally in fasted state.

In an embodiment, the administration of said abiraterone acetate tablet for oral suspension or abiraterone acetate granules for oral suspension provides peak abiraterone plasma concertation (Cₘₐₓ) which is about 129 to about 362 ng/mL when 187.5 mg therapeutically effective amount of abiraterone acetate administered orally in fasted state.

In an embodiment, the administration of said abiraterone acetate tablet for oral suspension or abiraterone acetate granules for oral suspension provides peak abiraterone plasma concertation (Cₘₐₓ) which is about 248 to about 662 ng/mL when 250 mg therapeutically effective amount of abiraterone acetate administered orally in fasted state.

In an embodiment, the administration of said abiraterone acetate tablet for oral suspension or abiraterone acetate granules for oral suspension provides peak abiraterone plasma concertation (Cₘₐₓ) which is about 61 to about 190 ng/mL when 250 mg therapeutically effective amount of abiraterone acetate administered orally in fed state.

In an embodiment, the administration of said abiraterone acetate tablet for oral suspension or abiraterone acetate granules for oral suspension provides abiraterone exposure (AUC) which is about 53 to about 144 h*ng/mL when 62.5 mg therapeutically effective amount of abiraterone acetate administered orally in fasted state.

In an embodiment, the administration of said abiraterone acetate tablet for oral suspension or abiraterone acetate granules for oral suspension provides abiraterone exposure (AUC) which is about 158 to about 409 h*ng/mL when 125 mg therapeutically effective amount of abiraterone acetate administered orally in fasted state.

In an embodiment, the administration of said abiraterone acetate tablet for oral suspension or abiraterone acetate granules for oral suspension provides abiraterone exposure (AUC) which is about 314 to about 875 h*ng/mL when 187.5 mg therapeutically effective amount of abiraterone acetate administered orally in fasted state.

In an embodiment, the administration of said abiraterone acetate tablet for oral suspension or abiraterone acetate granules for oral suspension provides abiraterone exposure (AUC) which is about 500 to about 1350 h*ng/mL when 250 mg therapeutically effective amount of abiraterone acetate administered orally in fasted state.

In an embodiment, the administration of said abiraterone acetate tablet for oral suspension or abiraterone acetate granules for oral suspension provides abiraterone exposure (AUC) which is about 266 to about 761 h*ng/mL when 250 mg therapeutically effective amount of abiraterone acetate administered orally in fed state.

In one embodiment, the therapeutically effective amount of the abiraterone acetate in the form of tablet for oral suspension or granules for oral suspension is from about 62.5 mg/day to about 1000 mg/day.

In one embodiment, the therapeutically effective amount of the abiraterone acetate in the form of tablet for oral suspension or granules for oral suspension is from about 250 mg/day to about 1000 mg/day.

In an embodiment, the therapeutically effective amount of the abiraterone acetate in the form of tablet for oral suspension or granules for oral suspension is administered orally as one (1) of 250 mg tablet for oral suspension or one (1) of 250 mg sachets containing granules for oral suspension once a day or 2 times a day (BID) or 3 times a day (TID) or 4 times a day (QID).

In an embodiment, the therapeutically effective amount of the abiraterone acetate in the form of tablet for oral suspension or granules for oral suspension is administered orally as one (1) of 250 mg tablet for oral suspension or one (1) of 250 mg sachets containing granules for oral suspension 2 times a day (BID).

In an embodiment, the therapeutically effective amount of the abiraterone acetate in the form of tablet for oral suspension or granules for oral suspension is administered orally as one (1) to four (4) of 62.5 mg abiraterone acetate tablet for oral suspension or one (1) to four (4) of 62.5 mg sachets containing abiraterone acetate granules for oral suspension once a day or 2 times a day (BID) or 3 times a day (TID) or 4 times a day (QID).

In an embodiment, the therapeutically effective amount of the abiraterone acetate in the form of tablet for oral suspension or granules for oral suspension is administered orally as one (1) to four (3) of 62.5 mg abiraterone tablet for oral suspension or one (1) to four (3) of 62.5 mg sachets containing abiraterone acetate granules for oral suspension once a day.

Furthermore, described herein is a method for the treatment of prostate cancer in a mammal comprising administering an amount of about 62.5 mg/day to about 1000 mg/day of abiraterone acetate.

In one embodiment the method for the treatment of prostate cancer in a mammal comprises the administration of an amount of about 62.5 mg/day to about 1000 mg/day of abiraterone acetate and a steroid.

In an embodiment, said steroid is chosen from hydrocortisone, hydrocortisone acetate, cortisone acetate, tixocortol pivalate, prednisolone, methylprednisolone, prednisone and dexamethasone.

### BRIEF DESCRIPTION OF THE DRAWINGS

FIG. 1 shows amorphous structure of HME milled granules (intragranular)

The following working Examples represent a preferred embodiment of the invention.

### EXAMPLES

### Example 1

Composition: Abiraterone acetate tablet for oral suspension—Prototype compositions of abiraterone acetate tablet for oral suspension formulations prepared are listed in Table 1 and Table 2.

TABLE 1
Compositions of abiraterone acetate tablet for oral
suspension formulations prepared

| | | Theoretical quantity | | | | | |
|---|---|---|---|---|---|---|---|
| | | Sub lot | Sub lot | Sub lot | Sub lot | Sub lot | Sub lot |
| | | 1 to 6 | 7 | 8 | 9 | 10 | 11 |
| Material | | % w/w | % w/w | % w/w | % w/w | % w/w | % w/w |
| 1 | Hot melt extruded (HME) | | | | | | |
| | milled granules (Intra- | | | | | | |
| | granular) | | | | | | |
| | Abiraterone acetate | 3.826 | 3.826 | 3.826 | 3.826 | 3.826 | 3.826 |
| | polyvinylpyrrolidone | 19.133 | 19.133 | 19.133 | 19.133 | 19.133 | 19.133 |
| | (Plasdone K12), | | | | | | |
| | Butylated hydroxytoluene | 0.025 | 0.025 | 0.025 | 0.025 | 0.025 | 0.025 |
| | (BHT) | | | | | | |
| | Butylated hydroxyanisole | 0.025 | 0.025 | 0.025 | 0.025 | 0.025 | 0.025 |
| | (BHA) | | | | | | |
| 2 | Sodium deoxy cholate | 2.3 | 2.3 | 2.3 | 2.3 | 2.3 | 2.3 |
| 3 | Polyvinylcaprolactam- | 15.31 | 15.31 | 15.31 | 15.31 | 15.31 | 15.31 |
| | polyvinyl acetate- | | | | | | |
| | polyethylene-glycol graft | | | | | | |
| | copolymers (Soluplus) | | | | | | |
| 4 | Lactose monohydrate | 23.27 | 22.49 | 22.89 | 22.89 | 21.99 | 20.99 |
| | (Flowlac 100) | | | | | | |
| 5 | Microcrystalline cellulose | 23.27 | 23.27 | 23.27 | 13.96 | 13.96 | 13.96 |
| | (Avicel PH101) | | | | | | |
| 6 | Microcrystalline cellulose | — | — | — | 9.31 | 9.31 | 9.31 |
| | (Vivapur 112) | | | | | | |
| 7 | Colloidal silicon dioxide | — | — | — | — | 0.5 | 0.5 |
| | (Aerosil 200) | | | | | | |
| 8 | Crospovidone (Kollidon CL) | 11.63 | 11.63 | 11.63 | 11.63 | 11.63 | 11.63 |
| 9 | Polyethylene glycol 4000 | 1.23 | 2 | 1.6 | 1.6 | 2 | 3 |

| | | | | | | |
|---|---|---|---|---|---|---|
| Total | 100 | 100 | 100 | 100 | 100 | 100 |

TABLE 2
Compositions of abiraterone acetate tablet for
oral suspension formulations prepared

| | | Theoretical quantity | | | |
|---|---|---|---|---|---|
| | | Sub lot | Sub lot | Sub lot | Sub lot |
| | | 12 | 13 | 14 | 15 |
| | Material | % w/w | % w/w | % w/w | % w/w |
| 1 | HME milled granules (Intra- | | | | |
| | granular) | | | | |
| | Abiraterone acetate | 3.826 | 3.826 | 3.826 | 3.826 |
| | polyvinylpyrrolidone | 19.133 | 19.133 | 19.133 | 19.133 |
| | (Plasdone K12), | | | | |
| | Butylated hydroxytoluene | 0.025 | 0.025 | 0.025 | 0.025 |
| | (BHT) | | | | |
| | Butylated hydroxy anisole | 0.025 | 0.025 | 0.025 | 0.025 |
| | (BHA) | | | | |
| 2 | Sodium deoxy cholate | 2.296 | 2.296 | 2.296 | 2.296 |
| 4 | Polyvinylcaprolactam-polyvinyl | 15.306 | 15.306 | 15.306 | 15.306 |
| | acetate-polyethylene-glycol graft | | | | |
| | copolymers (Soluplus) | | | | |
| 5 | Lactose monohydrate (Flowlac 100) | 21.563 | 21.363 | 21.363 | 21.563 |
| 6 | Microcrystalline cellulose (Avicel | 13.959 | 13.959 | 13.959 | 13.959 |
| | PH101) | | | | |
| 7 | Microcrystalline cellulose (Vivapur | 9.306 | 9.306 | 9.306 | 9.306 |
| | 112) | | | | |
| 8 | Colloidal silicon dioxide (Aerosil | 0.5 | 0.5 | 0.5 | 0.5 |
| | 200) | | | | |
| 9 | Crospovidone (Kollidon CL) | 11.633 | 11.633 | 11.633 | 11.633 |
| 10 | Aspartame powder | — | — | 0.2 | — |
| 11 | Sodium saccharine | — | 0.2 | — | — |
| 12 | Polyethylene glycol 4000 | 2 | 2 | 2 | 2 |
| 13 | Sodium sterayl fumarate | 0.428 | 0.428 | 0.428 | 0.428 |

| | | | | |
|---|---|---|---|---|
| Total | 100 | 100 | 100 | 100 |

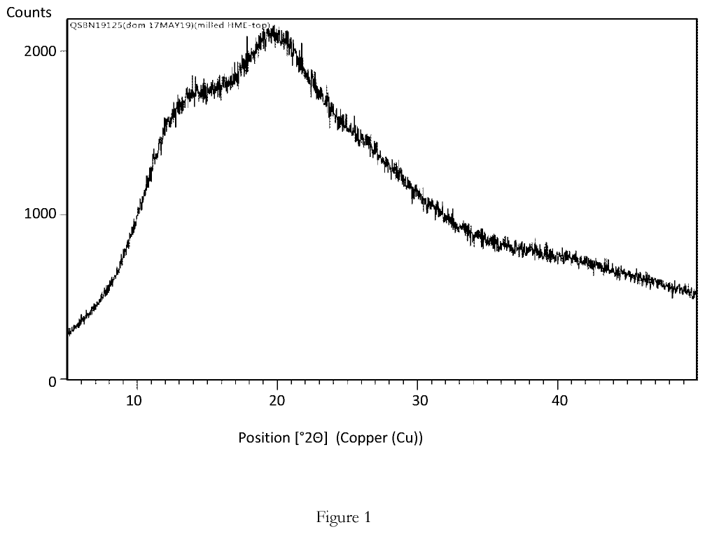

Disintegration, assay and related substance, dissolution, processability and crystallinity of the prepared compositions were investigated right after the production and 1 and 3 months later. Results are seen in Table 3-6 and FIG. 1.

TABLE 3
Disintegration of abiraterone acetate tablet for
oral suspension compositions prepared

| | | Mean | | |
|---|---|---|---|---|
| | Visual | Disintegration- | | |
| Sub | Disintegration | Apparatus | LOD (%) | |
| lot | (Sec) | (Sec) | Granules | Final Blend |
| 1A | 180 | 110 | ND | 3.53 |
| 1B | 150 | 87 | ND | 3.53 |
| 2 | ND | 117 | 2.5 | 2.78 |
| 3 | 175 | 97 | 2.31 | 2.69 |
| 4 | NA | NA | 2.31 | 3.15 |
| 5 | NA | NA | 2.45 | 2.93 |
| 6 | 180 | 145 | 2.38 | 2.97 |
| 7 | 78 | NP | NP | 2.7 |
| 8 | 77 | NP | NP | 3.67 |
| 9 | 148 | 138 | NP | 3.18 |
| 10 | 170 | 167 | NP | 2.59 |
| 11 | NA | NA | NP | 2.47 |
| 12 | 120 | 120 | NP | 2.41 |
| 13 | 170 | 165 | NP | 2.32 |
| 14 | 180 | 175 | NP | 1.98 |
| 15 | 124 | 125 | NP | 2.22 |

ND: Not detectable
NP: Not performed
NA: Not applicable

TABLE 4
Processability of tablet for oral suspension formulations prepared

| | | | Tablets | | | Mean |
|---|---|---|---|---|---|---|
| | Machine parameter | | (Yes/No) | | Friability | (N) |
| Sub lot | Weight | Thickness | compressed | Comments | (%) | Hardness |
| 1A | 8 | 3 | Yes | No issues | 1.13 | 88.27 |
| 1B | 8 | 3 | Yes | Sticking | 2.55 | 67.34 |
| 2 | 8 | 3 | Yes | Rat hole effect, | 0.84 | ND |
| | | | | Sticking | | |
| 3 | 8 | 3 | Yes | Sticking | 1.21 | 79.9 |
| 4 | 8 | 3 | No | Rat hole effect | NA | NA |
| 5 | 8 | 3 | No | Bridging effect | NA | NA |
| 6 | 8 | 3 | Yes | No issues | 0.81 | 95.3 |
| 7 | 8 | 3 | Yes | No issues | 1.52 | 67.5 |
| 8 | 8 | 3 | Yes | Sticking | 1.17 | 77.7 |
| 9 | 8 | 3 | Yes | Intense | 0.92 | 89.2 |
| | | | | Sticking | | |
| 10 | 8 | 3 | Yes | Minor sticking | 0.87 | 89.7 |
| 11 | 8 | 3 | No | Rat hole effect, | NA | NA |
| | | | | Sticking | | |
| 12 | 8 | 3 | Yes | No issues | 1.56 | 79.4 |
| 13 | 8 | 3 | Yes | No issues | 0.83 | 81.4 |
| 14 | 8 | 3 | Yes | No issues | 0.88 | 85.4 |
| 15 | 8 | 3 | Yes | No issues | 1.28 | 75.6 |

NA: Not applicable

The process was scaled-up and 36 Kg batch of sublot 13 was prepared.

9 Kg of intra-granular extrudate was prepared triturating BHT and BHA with a small portion of polyvinylpyrrolidone (Plasdone K12) and then screened and blended. More polyvinylpyrrolidone (Plasdone K12) was added to the mixture then blended again. The blend transferred to a 30 L Pharmatech blender and the residual amount of polyvinylpyrrolidone (Plasdone K12) as well as the abiraterone acetate was added into the blender shell then the material was blended again. A hot-melt extruder was set up according to the following parameters. Extrusion melt pressure was less than 300 PSI, Feed rate was 30 g/min (volumetric feeding), the temperature of all zones was 150° C. and the temperature of feed zone was 10° C. The Screw speed was set to 100 rpm and the die dimension was 1.0 mm. The extrudate was collected to an appropriate click lock container. The extrudated filament was milled with using a Quadro Comil having a 1143 μm screen at approximately 5,000 rpm speed. The appropriate amount of intragranular HME milled granules were dispensed for further process. Approximately 1 kg HME granules and the right amount of sodium deoxycholate, sodium saccharine and colloidal silicon dioxide were mixed manually then dispensed to a 30 L blender shell and blended together with the residual HE milled granules. The blend was transferred to a 100 L Pharmatech blender shell together with the appropriate amount of Soluplus, lactose monohydrate, microcrystalline cellulose (Avicel PH101), microcrystalline cellulose (Vivapur 112) and crospovidone then blended.

The polyethylene glycol 4000 and sodium stearyl fumarate and a small portion of the blend from the previous step were co-screened and transferred into the above 100 L blender shell then the 36 kg final tableting blend was lubricated.

The lubricated tableting blend was then compressed to tablets in a rotary press. The tablets were packed into an aluminum (PET/PE/Al/PE) pouch and heat sealed.

TABLE 5
Assay and related substance results of abiraterone acetate tablet for
oral suspension formulations prepared at different timepoints

| Storage time at | | |
|---|---|---|
| 40° C./75% RH | Assay (%) | Total related substances (%) |
| T = 0 | 98% | 0.1% |
| T = 1 month | 95% | 0.2% |
| T = 3 months | 98% | 0.5% |
| T = 6 months | 101% | 0.8% |
| T = 12 months | 96% | 0.9% |
| Storage time at | | |
| 25° C./60% RH | Assay (%) | Total related substances (%) |
| T = 0 | 98% | 0.1% |
| T = 1 month | 98% | 0.1% |
| T = 3 months | 100% | 0.2% |
| T = 6 months | 100% | 0.2% |
| T = 9 months | 102% | 0.3% |
| T = 12 months | 97% | 0.3% |
| T = 18 months | 100% | 0.7% |

TABLE 6
Dissolution of tablet for abiraterone acetate oral suspension
formulations prepared at different timepoints.
Dissolution (% Release)

| Storage time | | | | | | | | |
|---|---|---|---|---|---|---|---|---|
| at 40° C./ | 1 min | | 5 min | | 10 min | | 15 min | |
| 75% RH | Mean | Range | Mean | Range | Mean | Range | Mean | Range |
| T = 0 | 48 | 29-93 | 90 | 90-91 | 94 | 91-95 | 94 | 94-95 |
| T = 1 month | 50 | 46-58 | 96 | 94-98 | 97 | 95-98 | 96 | 95-99 |
| T = 3 months | 55 | 48-59 | 95 | 93-93 | 95 | 93-96 | 95 | 94-96 |
| T = 6 months | 67 | 57-75 | 93 | 92-94 | 93 | 92-94 | 93 | 91-95 |
| T = 12 months | 70 | 64-78 | 95 | 94-97 | 95 | 94-97 | 96 | 95-97 |
| Storage time | | | | | | | | |
| at 25° C./ | | | | | | | | |
| 60% RH | Mean | Range | Mean | Range | Mean | Range | Mean | Range |
| T = 0 | 48 | 29-93 | 90 | 90-91 | 94 | 91-95 | 94 | 94-95 |
| T = 1 month | 47 | 43-52 | 94 | 93-96 | 96 | 94-98 | 95 | 93-97 |
| T = 3 months | 52 | 48-60 | 96 | 95-96 | 96 | 95-97 | 96 | 94-97 |
| T = 6 months | 46 | 41-60 | 94 | 92-96 | 95 | 93-97 | 95 | 93-97 |
| T = 9 months | 49 | 42-59 | 94 | 92-97 | 94 | 92-95 | 95 | 93-98 |
| T = 12 months | 57 | 48-72 | 94 | 91-100 | 93 | 92-93 | 94 | 92-95 |
| T = 18 months | 40 | 32-47 | 93 | 91-95 | 94 | 92-95 | 94 | 92-96 |

### Composition: Abiraterone Acetate Granules for Oral Suspension

For the development of granules for reconstitution the same formulation composition as Sublot 13, presented in Table 2 was used for sachet product development.

9 Kg of intra-granular extrudate was prepared triturating BHT and BHA with a small portion of polyvinylpyrrolidone (Plasdone K12) and then screened and blended. More polyvinylpyrrolidone (Plasdone K12) was added to the mixture then blended again. The blend transferred to a 30 L Pharmatech blender and the residual amount of polyvinylpyrrolidone (Plasdone K12) as well as the abiraterone acetate was added into the blender shell then the material was blended again. A hot-melt extruder was set up according to the following parameters. Extrusion melt pressure was less than 300 PSI, Feed rate was 30 g/min (volumetric feeding), the temperature of all zones was 150° C. and the temperature of feed zone was 10° C. The Screw speed was set to 100 rpm and the die dimension was 1.0 mm. The extrudate was collected to an appropriate click lock container. The extrudated filament was milled with using a Quadro comil having a 1143 m screen at approximately 5,000 rpm speed. The appropriate amount of intragranular HME milled granules were dispensed for further process.

Approximately 1 kg HME granules and the right amount of sodium deoxycholate, sodium saccharine and colloidal silicon dioxide were mixed manually then dispensed to a 30 L blender shell and blended together with the residual HME milled granules. The blend was transferred to a 100 L Pharmatech blender shell together with the appropriate amount of Soluplus, lactose monohydrate, microcrystalline cellulose (Avicel PH101), microcrystalline cellulose (Vivapur 112) and crospovidone then blended.

The polyethylene glycol 4000 and half of the sodium stearyl fumarate and a small portion of the blend from the previous step were co-screened and transferred into the above 100 L blender shell then the 36 kg final tableting blend was lubricated.

The lubricated blend was then roller compacted using suitable process parameters in order to prepare granules.

The granules were transferred packed into the above 100 L blender shell.

The second half of the sodium stearyl fumarate was screened and transferred the above 100 L blender shell then the 36 kg final granules were lubricated.

The granules were then packed into an aluminum (PET/PE/Al/PE) pouch and heat sealed.

Lead granule composition was selected based on the process visual observations, physical and chemical assessment. During manufacturing smooth compaction and grinding, and no building up of the ribbons were observed.

Based on physical assessment optimum granules particle size with the least amount of fines generated was selected. The granules could be described as having
Mean particle size=641 μm
Particles <125 μm=22.04%
Fine particles <90 μm=15.35%

Based on analytical assessment assay, filter assay, reconstitution time and dissolution profile results were within the specifications.

TABLE 7
Assay and related substance results of granule for abiraterone acetate
granules for oral suspension formulations prepared at different timepoints

| Storage time at | | |
|---|---|---|
| 40° C./75% RH | Assay (%) | Total related substances (%) |
| T = 0 | 99% | 0.1% |
| T = 1 month | 101% | 0.2% |
| T = 3 months | 98% | 0.3% |
| Storage time at | | |
| 25° C./60% RH | Assay (%) | Total related substances (%) |
| T = 0 | 99% | 0.1% |
| T = 1 month | 102% | 0.1% |
| T = 3 months | 100% | 0.1% |

### Example 2

An experiment was undertaken to investigate the effect of antioxidants on the stability of abiraterone acetate when produced as a milled HME (hot melt extrusion) matrix. Abiraterone acetate (API) and PVP were present at a ratio of 1:4 in the extrudate.

The melt extruded samples were ground using an IKA A11 basic analytical mill (IKA-Werke GmbH & CO. KG, Staufen, Germany).

TABLE 8

| | Abiraterone acetate (%) | | | | | |
|---|---|---|---|---|---|---|
| Amount of | | Right | 2 | 1 | 2 | 3 |
| antioxidants / | | after | weeks, | month, | months, | months, |
| 300 mg API | Initial | extrusion | 40° C. | 40° C. | 40° C. | 40° C. |
| — | 99.93 | 99.44 | n./a. | 98.96 | 98.62 | 98.20 |
| 10 mg TPGS | | 99.83 | 99.69 | 99.76 | 99.59 | 99.53 |
| 0.4 mg BHT | | 99.71 | 99.72 | 99.76 | 99.65 | 99.60 |
| 0.8 mg BHT | | 99.76 | 99.67 | 99.82 | 99.61 | 99.69 |
| 1.6 mg BHT | | 99.78 | 99.73 | 99.82 | 99.66 | 99.72 |
| 2.5 mg BHA | | 99.76 | 99.74 | 99.81 | 99.69 | 99.72 |
| 1.6 mg BHT, | | 99.80 | n./a. | 99.74 | 99.81 | 99.77 |
| 1.6 mg BHA | | | | | | |
| 1.6 mg BHT, | | 99.75 | n./a. | 99.74 | 99.81 | 99.78 |
| 2.5 mg BHA | | | | | | |
| 1.6 mg BHT, | | 99.64 | n./a. | 99.79 | 99.66 | 99.57 |
| 10 mg BHA | | | | | | |

As can be seen, the absence of any antioxidant in the extruded composition results in a loss (degradation) of abiraterone acetate. However, when an optimal blend of BHA and BHT are present, the loss/degradation is negligible i.e. <0.3% after storage for 3 months at 40° C.

### Example 3

### Pharmacokinetics in Healthy Man

FDA considers an abiraterone acetate formulation equivalent to Zytiga® in case the main pharmacokinetic parameters (AUC, Cₘₐₓ) fall between the lower and upper limits defined as parameters determined for Zytiga® in the fasted and modified fasting state, respectively. Two clinical pharmacokinetic studies were designed to characterize the pharmacokinetic properties of the novel formulation in order to determine the dose regimen equivalent to 1000 mg daily Zytiga® and to investigate the effect of food on absorption characteristics.

Healthy subjects were selected by the investigators based on their medical history, physical examination, electrocardiograms and routine clinical laboratory test results. Twelve (C01) and twenty (C02) male subjects aged 21-65 years were enrolled. All subjects gave written informed consent and received an inconvenience allowance for their participation.

Study C01 (dose proportionality) and Study C02 (comparative food effect) were both single-dose, randomized, open-label, four-period, four-treatment crossover studies conducted at a single center at Quotient Clinical (Nottingham, UK) in accordance with the Clinical Protocol, with the Declaration of Helsinki and its amendments, with the International Conference on Harmonisation Good Clinical Practice (ICH GCP) Guidelines, and in accordance with all applicable regulatory requirements. The abiraterone acetate tablet for oral suspension was manufactured by hot melt extrusion and formulated as 62.5 mg unit dose tablets for oral suspension (as per sublot 13 above). The administered doses were prepared prior to dosing by the reconstitution of the tablet(s) in 240 ml of water. Zytiga® tablets were obtained commercially and were administered orally as four 250 mg tablets swallowed with 240 ml of water. In both studies venous blood samples of approximately 4 ml were collected for the determination of plasma concentrations of abiraterone immediately prior to dosing and at 0.25, 0.5, 0.75, 1, 1.5 2, 3, 4, 6, 8, 12, 16, 24, 48 and 72 hours post dose and pharmacokinetic parameters were calculated. The minimum washout period between the consecutive doses was at least 7 days.

In Study C01 subjects (n=12) were administered 1-4 abiraterone acetate tablets for oral suspension (corresponding to 62.5 mg, 125 mg, 187.5 mg and 250 mg of abiraterone acetate) in the fasted state. Study C02 (n=20) was a comparative study between 1000 mg Zytiga® and 250 mg abiraterone acetate tablet for oral suspension (four tablets) including the investigation of the effect of food on the pharmacokinetics of the tablet for oral suspension. Subjects were administered Zytiga® in the fasted state and in the modified fasting state (meal 2 hours before administration, as described in the Zytiga® label), while the tablet for oral suspension was administered in the fasted state and after an FDA standard high-fat breakfast which was consumed within 30 minutes prior to dosing.

Following the administration of increasing doses of abiraterone acetate tablet for oral suspension in the fasted state (C01 study) mean abiraterone plasma concentrations increased rapidly to peak and declined in a biphasic manner. Mean tₘₐₓ occurred within 1 hour regardless of dose. Dose proportional increases of Cₘₐₓ, and more, than dose proportional increases of AUC were observed. AUC and Cₘₐₓ values indicated that in the fasted state the pharmacokinetic parameters for the 250 mg abiraterone acetate tablet for oral suspension will fall between the lower and upper limits (Table 8).

Comparative investigations with Zytiga® (C02 study) were in agreement with this prediction. In the fasted state 1000 mg Zytiga® exhibited very low plasma concentrations, while in the modified fasting state AUC and Cₘₐₓ increased 6.5-times and 5.3-times, respectively. AUC and Cₘₐₓ for abiraterone acetate tablet for oral suspension fell between this range in the fasted state. A high-fat breakfast reduced AUC and Cₘₐₓ by 25% and 65%, respectively. As a result, while Cₘₐₓ was still in the range defined by the Zytiga® arms, AUC was slightly (˜10%) below the minimal AUC defined by 1000 mg Zytiga® when taken in the fasted state. (Table 9).

In conclusion, a single daily 250 mg dose of the abiraterone acetate tablet for oral suspension satisfies the FDA defined conditions for Zytiga® equivalent formulations when administered in the fasted state, while AUC falls ˜10% below the lower limit in the fed state. Repeat dose simulations indicate that trough concentrations are below 8.2 ng/ml, which was shown to be an important threshold for the pharmacological response to therapy. 250 mg BID dosing satisfies the equivalence criteria for both Cₘₐₓ and AUC in both the fasted and the fed states, while simulation of repeat BID dosing indicates that trough plasma levels will be 13-14 ng/ml. In summary, 250 mg BID is expected to deliver adequate and safe abiraterone plasma levels with more reliable abiraterone plasma concentrations when compared to Zytiga® without the need to take the drug on an empty stomach. Furthermore, the novel dosage form (water dispersible tablet) allows more convenient drug administration in patients with dysphagia or with problems with the large pill burden of taking four large Zytiga® tablets.

TABLE 8
Pharmacokinetic parameters (mean +/− SD) of abiraterone
following the oral administration of increasing doses of
abiraterone tablet for oral suspension in the fasted state.

| Dose (mg) | tₘₐₓ (h) | Cₘₐₓ (ng/ml) | AUCₗₐₛₜ (h*ng/ml) | T1/2 (h) |
|---|---|---|---|---|
| 62.5 | 0.73 ± 0.33 | 47.6 ± 26.9 | 97.4 ± 41.7 | 15.6 ± 2.0 |
| 125 | 0.70 ± 0.31 | 147.0 ± 101.1 | 261.6 ± 105.6 | 17.3 ± 5.7 |
| 187.5 | 0.68 ± 0.16 | 296.5 ± 166.4 | 544.5 ± 202.0 | 15.8 ± 5.8 |
| 250 | 0.86 ± 0.13 | 451.7 ± 190.1 | 913.1 ± 350.2 | 14.6 ± 7.6 |

TABLE 9
Pharmacokinetic parameters (mean +/− SD) of abiraterone following
the oral administration of 250 mg abiraterone acetate tablet for oral
suspension or 1000 mg Zytiga ® at different prandial states.

| | Abiraterone | Abiraterone | | |
|---|---|---|---|---|
| | acetate tablet | acetate tablet | | |
| | for oral | for oral | | Zytiga |
| | suspension | suspension | Zytiga | Modified |
| | Fasted | Fed | Fasted | Fasted |
| AUC(ng*h/ml) | 715 | 534 | 606 | 3926 |
| 2*AUC(ng*h/ml) | 1431 | 1070 | N.A. | N.A. |
| Cmax(ng/ml) | 381 | 140 | 119 | 632 |
| tmax(h) | 0.7 | 0.9 | 1.9 | 2.7 |

1-40. (canceled)

41. A solid dosage form for oral administration comprising a stable, amorphous matrix comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid, prior to ingestion.

42. The solid dosage form for oral administration according to claim 41, wherein said stable, amorphous matrix exhibits one or more of the following: (i) X-ray amorphous character in the solid form; (ii) a glass transition when assessed via differential scanning calorimetry (DSC), and (iii) is stable for at least 3 months at 40° C., as demonstrated by a lack of significant decomposition and/or oxidation and/or crystallization of abiraterone acetate.

43. The solid dosage form for oral administration according to claim 41, wherein said stable, amorphous matrix comprises one or more water-soluble polymers selected from: polyvinylcaprolactam-polyvinyl acetate-polyethylene-glycol graft copolymers, poloxamers; polyvinylpyrrolidone, poly(acrylic acid), polyvinyl alcohol, ethylene glycol and vinyl alcohol graft copolymer, hydroxypropyl methylcellulose, hydroxypropylcellulose, hydroxyethylcellulose, hypromellose acetate succinate, polyethylene oxide, polyethylene-glycol, poly(2-Ethyl-2-oxazoline), poly(methyl vinyl ether/maleic anhydride), poly(maleic acid-co-methyl vinyl ether, polyoxylglycerides, D-α-tocopherol polyethylene glycol 1000 succinate, polyoxyethylene castor oil derivatives, polyoxyethylene sorbitan fatty acid esters, polylactic acid, poly(lactic-co-glycolic acid) and copolymers of vinylpyrrolidone and vinyl-acetate and poly(maleic acid-co-methyl-vinyl-ether), preferably polyvinylpyrrolidone, such as K12 or Kollidon 17PF, Kollidon 25, Kollidon 30 or Kollidon 90, most preferably a K12 grade.

44. The solid dosage form for oral administration according to claim 41, wherein said stable, amorphous matrix comprises one or more antioxidants selected from: ascorbic acid, ascorbyl palmitate, butylated hydroxyanisole (BHA), butylated hydroxytoluene (BHA), hypophosphorous acid, monothioglycerol, potassium metabisulfite, propyl gallate, sodium bisulfite, sodium formaldehyde sulfoxylate, sodium metabisulfite, sodium sulfite, sodium thiosulfate and tocopherols, preferably BHA and/or BHT.

45. The solid dosage form for oral administration according to claim 41, wherein said one or more pharmaceutically excipients are selected from group consisting of: diluents or fillers, binders, disintegrants and dispersing agents, lubricants, glidants, antiadherents, surfactants, sweeteners and flavourings.

46. The solid dosage form for oral administration according to claim 45, wherein said diluents or fillers are selected from: pullulan, lactose (anhydrous), lactose monohydrate, mannitol, sucrose, glucose, plant cellulose, calcium carbonate, magnesium carbonate, magnesium oxide, microcrystalline cellulose, silicified microcrystalline cellulose, sorbitol, starch, pregelatinized starch, isomalt, anhydrous dibasic calcium phosphate, dibasic calcium phosphate dihydrate, calcium silicate, magnesium aluminium silicate, maltodextrin, dextrates; and/or
said binders are selected from: pullulan, cellulose, methyl cellulose, microcrystalline cellulose, cellulose ethers such as hydroxypropyl cellulose, polyvinylpyrrolidone, polyethylene glycol, lactose, sucrose, mannitol, sorbitol and xylitol and their co-processed versions such as alpha-Lactose-monohydrate and cellulose or starch, urea crystals, sodium sulfate and calcium sulfate dehydrate;
said disintegrants and dispersing agents are selected from: polyvinylpyrrolidone, crosslinked polyvinylpyrrolidone (crospovidone), polyvinylcaprolactam-polyvinyl acetate-polyethylene-glycol graft copolymers, carboxymethyl cellulose, crosslinked cellulose and its sodium salt (crosscarmellose), crosslinked sodium carboxymethyl cellulose, modified starch, sodium starch glycolate, crosslinked starch, crosslinked alginic acid and sodium starch glycolate, colloidal silicon dioxide, soy polysaccharides and sodium deoxycholate; and/or
said lubricants are selected from: polyethylene glycol, calcium stearate, silica, talc, stearic acid, sodium stearyl fumarate, sodium lauryl sulfate, sodium benzoate, stearic acid and magnesium stearate; and/or
said glidants are selected from: silica gel, colloidal silicon dioxide, fumed silica, talc and magnesium carbonate; and/or
said antiadherents are selected from: magnesium stearate, talc and starch; and/or
said surfactants are selected from: polysorbates such as Tweens, polysorbates, Sodium dodecyl sulfate (sodium lauryl sulfate), lauryl dimethyl amine oxide, docusate sodium, cetyl trimethyl ammonium bromide (CTAB), polyethoxylated alcohols, polyoxyethylene sorbitan, octoxynol, N,N-dimethyldodecylamine-N-oxide, hexadecyltrimethylammonium bromide, polyoxyl 10 lauryl ether, Brij, bile salts (sodium deoxycholate, sodium cholate), polyoxyl castor oil, nonylphenol ethoxylate cyclodextrins, lecithin, methylbenzethonium chloride, petroleum sulphonates, alkylbenzenesulphonates, sulphated alkanolamides, polyoxyethylene surfactants, carboxylic esters, polyethylene glycol esters, glycol esters of fatty acids, carboxylic amides, quaternary ammonium salts, preferably docusate sodium and/or sodium lauryl sulfate; and/or
said sweeteners are selected from: sodium saccharine, sucrose, saccharine, aspartame, acesulfame-K, sodium cyclamate and sorbitol.

47. The solid dosage form for oral administration according to claim 41, wherein said abiraterone acetate is present in said stable, amorphous matrix at an amount ranging from about 1.0% by weight to about 95.0% by weight, based on the total weight of the stable, amorphous matrix.

48. The solid dosage form for oral administration according to claim 41, wherein said stable, amorphous matrix is present at an amount ranging from about 1.0% by weight to about 95.0% by weight, based on the total weight of the solid dosage form, preferably wherein said stable, amorphous matrix is present in particulate form, preferably as a particulate or milled extrudate.

49. The solid dosage form for oral administration according to claim 41, wherein said solid dosage form is selected from: a tablet, coated tablet, effervescent tablet, tablet for oral suspension, fast melt tablet, lyophilized tablet, lyophilized wafer, disintegrating tablet, dispersible tablet, orodispersible tablet, mini-tablet, multilayer tablet, bi-layered tablet, tablet-in-tablet, pill, micro-pellet, small tablet unit, powder, powder for reconstitution, powder for oral suspension, pellets, beads, MUPS (multiple unit pellet system), granules, dry granules, roller compacted granules, effervescent granules, granules for oral suspension, slugs, microspheres, multiparticulates, sprinkles, preferably granules for oral suspension, wherein said solid dosage form is preferably packaged as a blister, bottle, capsule, stick or sachet.

50. The solid dosage form for oral administration according to claim 49, wherein said micro-pellet, small tablet unit, powder, powder for reconstitution, powder for oral suspension, pellets, beads, MUPS (multiple unit pellet system), granules, dry granules, roller compacted granules, effervescent granules, granules for oral suspension, slugs, microspheres, multiparticulates and sprinkles preferably have a D50 of greater than 300 micron and where less than about 10% by weight of the total mass of the solid dosage form is less than 63 microns, preferably wherein less than about 10% by weight of the total mass of the solid dosage form is less than 50 microns, as measured by laser diffraction or preferably sieve fractionation.

51. The solid dosage form for oral administration according to claim 41, wherein said solid dosage form is presented as one of the following: controlled release formulation, immediate release formulation, fast melt formulation, lyophilized formulations, delayed release formulation, extended release formulation, pulsatile release formulation, or mixed immediate release and controlled release formulation.

52. The solid dosage form for oral administration according to claim 41, which comprises a therapeutically effective amount of abiraterone acetate, ranging from about 10 mg to 2000 mg per dosage form, or about 50 mg to 1000 mg per dosage form, preferably about 62.5 mg to about 250 mg per dosage form.

53. The solid dosage form for oral administration according to claim 41, wherein said solid dosage form disintegrates and/or releases abiraterone acetate into an aliquot of liquid suitable for human consumption.

54. The solid dosage form for oral administration according to claim 53, wherein said aliquot of liquid suitable for human use is selected from: water, fruit juice such as orange juice, apple juice, pineapple juice or cranberry juice, coconut water, coconut milk, almond milk, oat milk, soya milk, rice milk, and dairy-derived milks such as cow's milk.

55. The solid dosage form for oral administration according to claim 53, wherein said solid dosage form disintegrates and/or releases at least 85% or more of said abiraterone acetate into said aliquot of liquid in less than about 10 minutes, preferably less than about 5 minutes, more preferably less than about 3 minutes, or less than about 1 minute or less, wherein said aliquot is 250 ml or less, such as about 50 or 60 ml.

56. The solid dosage form for oral administration according to claim 41, wherein said abiraterone acetate is present in said solid dosage form at about 1.0% by weight to about 20.0% by weight, or about 1.0% to about 10.0% by weight, preferably about 1.0% to about 5.0% by weight, based on the total weight of the solid dosage form.

57. A solid dosage form for oral administration comprising: (a) abiraterone acetate; (b) one or more water soluble polymers, and (c) one or more antioxidants, wherein said solid oral dosage form preferably further comprises a disintegrant and one or more pharmaceutically acceptable excipients, and wherein said solid dosage form for oral administration is capable of rapid reconstitution or disintegration in the oral cavity or an aliquot of liquid prior to ingestion, comprising:
(a) 1-20% abiraterone acetate,
(b) 5-60% water soluble polymer,
(c) 35-90% fillers and/or diluents,
(d) 5-20% binders,
(e) 5-45% disintegrants,
(f) 0.1-5% lubricants,
(g) 0.1-5% antiadherents,
(h) 0.1-5% glidants,
(i) 0.01-2% antioxidants, and
(j) 0.05-5% flavouring agents and/or sweeteners.

58. The solid dosage form for oral administration according to claim 41, for use in the treatment of prostate cancer.

59. Use of a solid dosage form for oral administration according to claim 41, in the manufacture of a medicament for the treatment of prostate cancer.

60. A method of producing a solid dosage form for oral administration comprising abiraterone acetate, wherein said solid dosage form for oral administration is capable of rapid reconstitution or disintegration in the oral cavity or an aliquot of liquid prior to ingestion, wherein said method comprising the steps of:
(a) providing abiraterone acetate;
(b) providing one or more water-soluble polymers, preferably polyvinylpyrrolidone, such as K12 or Kollidon 17PF, Kollidon 25, Kollidon 30 or Kollidon 90, most preferably a K12 grade;
(c) providing one or more antioxidants;
(d) processing (a), (b) and (c) by blending to produce powder blend comprising (a) to (c);
(e) processing the product of step (d) by an extrusion process to produce an extruded composition;
(f) processing the extruded composition of step (e) by size reduction such as milling, to produce a particulate or milled extrudate;
(g) blending the particulate or milled extruded composition of step (f) with one or more pharmaceutically acceptable excipients such as fillers, diluents, binders, disintegrants, lubricants, antiadherents, glidants, flavoring agents and sweeteners to produce a composition blend; and
(h) compressing the composition blend of step (g) into a solid dosage form such as via direct compression to form a tablet, or, preparing granules from the composition blend of step (g), via slugging and/or milling and/or roller compaction to produce such granules.
